(19) United States
(12) Patent Application Publication
(10) Pub. No.: US 2019/0022220 A1
(43) Pub. Date: Jan. 24, 2019

(54) LIGHT-ACTIVATED ANCHORING OF THERAPEUTIC FACTORS TO TISSUES

(71) Applicants: The Board of Trustees of the Leland Stanford Junior University, Stanford, CA (US); U.S. GOVERNMENT AS REPRESENTED BY THE DEPARTMENT OF VETERANS AFFAIRS, Washington, DC (US)

(72) Inventors: Jeffrey Goldberg, Palo Alto, CA (US); David Myung, San Jose, CA (US)

(21) Appl. No.: 16/071,052

(22) PCT Filed: Jan. 29, 2017
(86) PCT No.: PCT/US2017/015531

§ 371 Date: Jul. 18, 2018

Related U.S. Application Data
(60) Provisional application No. 62/289,242, filed on Jan. 30, 2016.

(51) Int. Cl. A61K 41/00; A61P 17/02; A61K 9/08; A61K 9/06; A61L 15/44; A61L 26/00; A61L 15/28; A61K 31/728; A61K 47/61; A61K 45/06; A61K 38/18; A61K 38/39; A61K 47/22; A61K 35/545; A61B 17/32; A61N 5/06; A61F 9/00

(52) U.S. Cl. CPC A61K 41/0057; A61N 2005/0667; A61K 9/08; A61K 9/06; A61L 15/44; A61L 26/0066; A61L 26/0023; A61L 15/28; A61K 31/728; A61K 47/61; A61K 45/06; A61K 38/1808; A61K 38/185; A61K 38/39; A61K 47/22; A61K 35/545; A61B 17/32; A61N 5/062; A61F 9/0017; A61L 2430/34; A61L 2300/402; A61L 2300/41; A61L 2300/404; A61L 2300/416; A61L 2300/42; A61B 2017/320004; A61N 2005/0661; A61N 2005/0663; A61P 17/02

(57) ABSTRACT

Compositions and methods for repairing or regenerating damaged tissue are disclosed. In particular, the invention relates to methods of anchoring biomolecules and/or cells to tissues in order to immobilize and concentrate therapeutic factors that promote tissue regeneration at or under the surface of damaged tissue.

### TECHNICAL FIELD

The present invention pertains generally to compositions and methods for repairing or regenerating damaged tissue. In particular, the invention relates to methods of anchoring biomolecules and/or cells to tissues in order to immobilize and concentrate therapeutic factors that promote tissue regeneration at or under the surface of damaged tissue.

### BACKGROUND

The following discussion of the background art is intended to facilitate an understanding of the present invention only. The discussion is not an acknowledgement or admission that any of the material referred to is or was part of the common general knowledge as of the priority date of the application.

Tissue regeneration is a complex process involving the temporal and spatial interplay between cells and their extracellular milieu. It can be impaired by a variety of causes including infection, poor circulation, loss of critical cells and/or proteins, and a deficiency in normal neural signaling such as in neurotrophic ulcers (Suzuki et al. (2003) Prog. Retin. Eye Res. 22(2):113-133; Tran et al. (2004) Wound Repair and Regeneration 12(3):262-268). Moreover, uncontrolled wound responses can lead to scarring and contracture (Schultz et al. (2009) Wound Repair and Regeneration 17(2):153-162; Klenkler et al. (2007) The Ocular Surface 5(3):228-239). Ocular and peri-ocular anatomy is particularly vulnerable to severe morbidity, whether it be opacification of the cornea, residual deficits in cranial nerves, or cicatricial changes to the eyelids and adnexa.

Cell based therapies such as stem cell transplantation typically provide only cells without the required matrix upon which to grow, or without the stimulatory factors to which to respond by migration, proliferation, and/or differentiation. Topical approaches to wound healing have been reported using epidermal growth factor, thymosin beta 4, nerve growth factor, substance P and insulin-like growth factor, and fibronectin. However, a clinically proven biopharmacologic therapy has not yet been successfully developed.

Neurotrophic keratopathy (NK) is a degenerative disease of the cornea resulting from trigeminal nerve damage caused by a variety of conditions including diabetes, herpes, neoplasms, or trauma (Bonini et al. (2003) Eye 17(8):989-995; Dunn et al. (2010) Ann NY Acad Sci 1194(1):199-206). It is hallmarked by decreased corneal sensitivity, reduced reflex tearing, and poor wound healing, leaving the cornea susceptible to injury and progressive breakdown (Bonini et al., supra; Dunn et al., supra). NK poses a particularly difficult clinical challenge due to the limited efficacy of current treatments such as frequent lubrication, antibiotic drops or ointment, patching, and bandage contact lenses. In refractory cases, oral doxycycline, autologous serum, and application of an amniotic membrane, a flap of conjunctival tissue, or tarsorraphy are used alone or in combination (Abelson et al. (2014) Thoughts on Healing the Wounded Cornea, Review of Ophthalmology 2014; September:52-54). Amniotic membranes in particular have shown promising results, but wound closure times are still reported to be two weeks or greater (Kruse et al. (1999) Ophthalmology 106(8):1504-1511; Chen et al. (2000) Br. J. Ophthalmol. 84(8):826-833). Despite the arsenal of modalities available, a protracted clinical course is often required and the healing response can be erratic (Abelson et al., supra), leaving the cornea at risk of infection, scarring, perforation, and blindness (Abelson et al., supra; Nagano et al. (2003) Invest Ophthalmol Vis Sci 44(9):3810-3815).

Corneal epithelial health is modulated by endogenous neuropeptides supplied by corneal nerves (Bonini et al. (2003) Eye 17(8):989-995). Promising yet limited results have been reported on the therapeutic potential of various topically applied neuropeptides and growth factors (Bonini et al., supra; Dunn et al., supra; Nagano et al., supra; Bonini et al. (2000) Ophthalmology 107(7):1347-1351). For instance, exogenous application of the neuropeptide Substance P (SP) has been shown to improve wound healing in NK, but its effects are enhanced when combined with another trophic agent such as epidermal growth factor (Guaiquil et al. (2014) Proc Natl Acad Sci USA 111(48):17272-17277). Topical neuroregenerative ligands such as nerve growth factor (NGF) have been shown in clinical trials to restore corneal innervation (Aloe et al. (2008) Pharmacological Research 57(4):253-258; Guaiquil et al., supra), but treatment requires four times daily administration and anywhere from 9 days to 6 weeks for wound closure to occur (Aloe et al. (2012) J. Transl. Med. 10:239). Recently, vascular endothelial growth factor (VEGF) has been shown in an animal model to stimulate regeneration of injured corneal nerves (Guaiquil et al., supra), but these results have not yet been reported in humans. Thus, to date, a clinically available, rapid-onset biopharmacologic therapy for NK remains elusive.

Thus, there remains a need in the art for better ways to stimulate a regenerative response in order to foster wound healing and restore anatomy and, in turn, tissue functions such as epithelial barrier effects and neural transmission.

### SUMMARY

The present invention relates to methods of crosslinking therapeutic factors to tissues in order to immobilize and concentrate therapeutic factors that promote wound healing at or under the surface of damaged tissue.

In one aspect, the invention includes a method of treating damaged tissue in a subject, the method comprising: a) contacting the damaged tissue with effective amounts of a photosensitizer and one or more therapeutic factors capable of promoting tissue regeneration or repair; and b) exposing the tissue to light to induce a photocrosslinking reaction, wherein the one or more therapeutic factors are crosslinked directly to the damaged tissue and to one another.

Therapeutic factors that can be used in the practice of the invention include any biomolecule, drug, or cell, which when administered in combination with a photosensitizer as described herein, brings about a positive therapeutic response in treatment of damaged tissue, such as improved wound healing or tissue repair or regeneration. An effective amount of a therapeutic factor, for example, may accelerate healing of the damaged tissue, increase thickness of an epithelial layer of the damaged tissue, increase rate of epithelialization at the site of damaged tissue, shorten the time required for wound closure, or promote nerve regeneration in the damaged tissue. Therapeutic factors may include biomolecules (e.g., growth factors, neurotrophic factors, and extracellular matrix proteins), cells (e.g., stem cells), or a combination thereof. Exemplary biomolecules that can be used include growth factors (e.g., epidermal growth factor (EGF), nerve growth factor (NGF), vascular endothelial growth factor (VEGF), and insulin-like growth factor (IGF)), neuropeptides (e.g., substance P (SP)), extracellular matrix proteins (e.g., fibronectin, collagen, laminin, or fibrin), beta thymosins (thymosin beta-4), and netrins (netrin-1). Additionally, therapeutic factors may include antibiotic agents, antifibrotic agents, anti-inflammatory agents, chemotherapeutic (anti-oncologic) agents, anti-angiogenic agents, anti-thrombotic agents, or pro-thrombotic agents.

In certain embodiments, the photosensitizer is selected from the group consisting of riboflavin, rose bengal, and a phenyl azide compound, which require light to initiate a photochemical crosslinking reaction. In certain embodiments, the photosensitizer further includes a crosslinking moiety that does not require light to initiate a crosslinking reaction. Exemplary crosslinking moieties that do not require light include N-hydroxysuccinimide, dimethyl suberimidate, formaldehyde, and carbodiimide. In one embodiment, the method further comprises crosslinking a biomolecule with a photosensitizer via a non-light-activatable crosslinking moiety to produce a light-activatable bioconjugate of the biomolecule.

In certain embodiments, the photosensitizer and one or more therapeutic factors are applied to the damaged tissue at a surface or a subsurface. For example, the photosensitizer and one or more therapeutic factors may be applied at the surface of tissue (e.g., to promote wound closure) or beneath the surface (e.g. in stromal or subcutaneous tissue). In one embodiment, the photosensitizer and one or more therapeutic factors are applied at the location of a damaged nerve (e.g., to promote nerve regeneration).

The photosensitizer and one or more therapeutic factors may be contained in the same composition or separate compositions and may be applied to the damage tissue simultaneously or sequentially. The photosensitizer can be applied to the damaged tissue before or after one or more of the therapeutic factors. Different therapeutic factors may be applied to the damaged tissue simultaneously or separately. Furthermore, crosslinking of different therapeutic factors to the damaged tissue can be performed with the same photosensitizer or different photosensitizers.

Upon exposure to light (e.g., UV or visible), the photosensitizer reacts with surrounding molecules, including the therapeutic factors and the proteins of the tissue, resulting in crosslinking (i.e., formation of direct bonds) between the therapeutic factors and the tissue and the therapeutic factors among one another. Biomolecules, for example, may include more than one functional group that can be crosslinked to allow formation of bonds among multiple biomolecules and a tissue surface or sub surface.

In certain embodiments, one or more therapeutic factors are applied to the damaged tissue in a pattern, tracks, or a gradient. For example, a gradient of growth factors or axon guidance factors can be used, e.g., to guide cell migration or nerve regeneration. A gradient can be produced, for example, by varying light intensity, the length of light exposure, or the concentration of biomolecules along the damaged tissue.

In certain embodiments, the method further comprises treating the subject with one or more other drugs or agents, such as, but not limited to, antibiotic agents, antifibrotic agents, anti-inflammatory agents, chemotherapeutic (anti-oncologic) agents, anti-angiogenic agents, or anti-thrombotic agents, pro-thrombotic agents, and analgesic or anesthetic agents.

In other embodiments, the method further comprises administering cellular therapy to the damaged tissue, which may include allogeneic cell therapy, autologous cell therapy, or stem cell therapy.

Any appropriate mode of administration may be used for treating damaged tissue in a subject. In certain embodiments, compositions comprising one or more therapeutic factors and/or photosensitizers are administered topically, subcutaneously, by injection or infusion. The compositions may be administered locally to a wound or adjacent to a wound. In one embodiment, a wound dressing comprising one or more therapeutic factors and/or photosensitizers is applied to the damaged tissue. The wound dressing may comprise, for example, a gel, a viscoelastic solution, putty, a physical matrix or a membrane.

Compositions comprising photosensitizers and/or therapeutic factors may take the form of a solution or gel. Moreover, the photocrosslinking reaction may change the viscosity of a composition. Additionally, compositions may further comprise a pharmaceutically acceptable excipient.

In certain embodiments, the tissue damage comprises a diabetic ulcer, a neurotrophic ulcer, a burn, a chemical injury, a nerve injury, or damage to corneal tissue (e.g., neurotrophic keratopathy, recurrent corneal erosion, a corneal ulcer, exposure keratopathy, or physical trauma).

In certain embodiments, the method further comprises preparing the damaged tissue prior to treating the subject by exfoliation or debridement of fibrotic or necrotic tissue.

In certain embodiments, multiple cycles of treatment are administered to the subject for a time period sufficient to effect at least a partial healing of the damaged tissue or more preferably, for a time period sufficient to effect a complete healing of the damaged tissue or wound closure.

In another aspect, the invention includes a method of treating damaged corneal tissue in a subject, the method comprising: a) contacting the damaged corneal tissue with effective amounts of a photosensitizer and one or more biomolecules capable of promoting tissue regeneration or repair, wherein the biomolecules are selected from the group consisting of epidermal growth factor (EGF), nerve growth factor (NGF), substance P (SP), insulin-like growth factor 1 (IGF-1), and netrin-1; b) exposing the tissue to light (e.g., UV or visible light) to induce a photocrosslinking reaction, whereby the one or more biomolecules are crosslinked directly to the damaged corneal tissue and to one another. This method can be used to treat a subject who has corneal tissue damage caused, for example, by neurotrophic keratopathy, recurrent corneal erosion, a corneal ulcer, exposure keratopathy, or physical trauma. The method may further comprise applying a non-UV absorbing contact lens to the cornea to limit a UV photocrosslinking reaction to the corneal surface or a bandage contact lens to the cornea after the photocrosslinking reaction. In one embodiment, the visible light is blue light.

In certain embodiments, the method comprises using at least two biomolecules. In one embodiment, the biomolecules comprise SP and EGF. In another embodiment, the biomolecules comprise NGF, SP and EGF. In another embodiment, the biomolecules comprise netrin-1, SP, and IGF-1.

In certain embodiments, one or more biomolecules are applied to the damaged corneal tissue in a pattern, tracks, or a gradient. For example, a gradient of growth factors or axon guidance factors can be used, e.g., to guide cell migration or nerve regeneration. A gradient can be produced, for example, by varying light intensity, the length of light exposure, or the concentration of biomolecules along the damaged corneal tissue. In some embodiments, the gradient comprises one or more biomolecules selected from the group consisting of EGF, SP, NGF, and netrin-1.

In another aspect, the invention includes a tissue repair system comprising: a) a UV or visible light source; b) one or more biomolecules selected from the group consisting of epidermal growth factor (EGF), nerve growth factor (NGF), substance P (SP), insulin-like growth factor 1 (IGF-1), and netrin-1; and c) a photosensitizer. In one embodiment, the photosensitizer is covalently coupled to a biomolecule. In certain embodiments, the tissue repair system further comprises a non-UV absorbing contact lens, a UV filter, or a topical applicator or dispenser. In another embodiment, the light source provides blue visible light. In another embodiment, the photosensitizer is riboflavin.

In certain embodiments, the tissue repair system comprises at least two biomolecules. In one embodiment, the tissue repair system comprises the biomolecules SP and EGF. In another embodiment, the tissue repair system comprises the biomolecules NGF, SP and EGF. In another embodiment, the tissue repair system comprises the biomolecules netrin-1, SP, and IGF-1.

In another aspect, the invention includes a kit comprising a tissue repair system, as described herein.

In another aspect, the invention includes a photosensitizer and a biomolecule for use in treating damaged tissue in a subject by photocrosslinking the biomolecule directly onto the damaged tissue.

These and other embodiments of the subject invention will readily occur to those of skill in the art in view of the disclosure herein.

### BRIEF DESCRIPTION OF THE FIGURES

FIGS. 1A and 1B show schematics illustrating the synthesis of a matrikine-like biomolecular assembly applied to a cornea. FIGS. 1A and 1B show the use of riboflavin to photoactivate a heterocrosslinking reaction (using visible or UV light) between corneal stroma and a mixture of a first biomolecule such as a matrix protein (e.g. collagen, fibronectin, or laminin), a second biomolecule such as a growth factor (e.g. EGF or NGF), and a third biomolecule such as neuropeptide (e.g. substance P) to enable both adhesion and proliferation of nearby epithelial cells. Other biomolecules and combinations of biomolecules can be used as shown in these schematics. Other photosensitizers or chemicals can be used to replace riboflavin. Other forms of light aside from UV light, such a visible white light or blue light (˜458 nm) may also be used. In addition, non-photochemical means can be used to facilitate the biomolecule-to-tissue reaction. This technology can be applied to issues other than the cornea. It may also be applied to the immobilization of biomolecules between the surface of a tissue.

FIGS. 2A and 2B show schematics depicting the two approaches being taken to treat neurotrophic keratopathy. FIG. 2A shows the use of a photochemical linker to immobilize biomolecules (e.g., EGF and the neuropeptide Substance P) to the cornea to stimulate epithelial healing. FIG. 2B shows the use of a neurotrophic factor such as Nerve Growth Factor (NGF) to the cornea in order to stimulate nerve regeneration and, in turn, endogenous neuropeptide secretion.

FIG. 3 shows fluorometry data showing increased fluorescence intensity on collagen coated glass substrates (above intrinsic autofluorescence of glass and collagen) with visible (blue) light-based crosslinking (V-CXL) of EGF-FITC to collagen coated surfaces.

FIG. 4 shows real-time SPR data showing (I) N-hydroxysuccinimide (NETS) functionalization of gold, (II) coupling of collagen to gold, (III) blocking of excess NHS with ethanolamine, (IV) visible light photochemical coupling of NGF to collagen, and (V) washing of excess NGF.

FIG. 5 shows ellipsometry data showing relative layer thickness of (I) unmodified glass, (II) covalently bound collagen, (III) physisorbed NGF on collagen, and (IV) photochemically bound NGF on collagen. The light gray bars show the change in relative thickness upon exposure to NGF receptor, showing greater NGF binding in the case of photochemically bound NGF.

FIG. 6 shows ELISA quantification of surface concentration (in pg/cm²) of EGF as a function of blue light exposure time using riboflavin-based CXL. Exposure times were varied by total time (2.5 seconds to 60 seconds) using either pulsed or constant exposure. The pulsed regimen involved 1 second on and on second off, so 5 seconds of pulsed exposure results in 2.5 seconds of total blue light exposure. In these experiments, EGF of 0.01 mg/ml and 0.001 mg/ml were applied, with riboflavin at either 0.025 mM or 0.25 mM followed by blue light (˜458 nm) exposure at either 300 mW/cm² or 100 mW/cm² for 5, 10, 20, 40, or 60 seconds, with pulsed regimens of 5, 10, and 20 seconds. The results show that EGF surface binding for higher intensity blue light and lower concentration of riboflavin is generally optimized at shorter and pulsed exposure regimens.

FIG. 7 shows a Western blot detecting applied NGF-FITC within corneal stroma: (I) NGF-FITC control (solution only) (II) topically applied NGF-FITC on corneal stroma, (III) non-photochemical attachment of NGF-FITC to corneal stroma, and (IV) and V-CXL coupling of NGF-FITC to corneal stroma. The presence of the higher molecular weight (MW) band is indicative of binding of the growth factor to the collagen, creating a larger macromolecular complex that is labeled with FITC as a result of the crosslinking.

FIG. 8 shows a bar chart of the normalized band intensity as a function of coupling strategy, showing that V-CXL and non-photochemical crosslinking provides higher NGF surface concentration on corneal stroma than topical delivery alone.

FIG. 9A shows EGF release from collagen gels formed by V-CXL, upon exposure to phosphate buffered saline (PBS) or 0.1% vs. 0.2% collagenase in PBS. Release from the gel is slow in the absence of collagenase, and is accelerated in a dose-dependent manner by collagenase activity. FIG. 9B shows rheology data showing gelation of the collagen using V-CXL, as noted by the substantial increase in the storage modulus over physical collagen gels. FIG. 9C summarizes the storage and loss modulus of collagen gels crosslinked using riboflavin and blue light exposure of different time intervals. FIG. 9D shows a tissue section of corneal stroma with FITC-labeled collagen gel formed by V-CXL using riboflavin and blue light exposure covalently binding it to the surface.

FIG. 10A shows live-dead assays showing % living human mesenchymal stem cells (hMSCs) when encapsulated within collagen gels formed by direct exposure to blue light in the presence of different concentrations of riboflavin, showing greater than 90% viability at 72 hours for concentrations of 0.25 mM or less for 20 sec exposure time at 100 mW/cm². FIG. 10 B shows that for the 0.025 mM riboflavin concentration and 20 sec exposure time, greater than 99% of the hMSCs remain viable 1 week after exposure, indicated excellent biocompatibility of the crosslinking regimen in the presence of living cells.

FIG. 11 shows cell seeding on EGF-bound collagen surfaces yielded greater proliferation of senescent primary CECs over 5 days compared to surfaces without chemically bound EGF.

FIG. 12A shows a plot showing the relationship between blue light intensity and distance from the cornea. We have found that the optimal intensity for V-CXL to couple growth factors is 60 mW/cm², where the blue light source is held 4 cm from the corneal wound, with optimal exposure time being approximately 2.5 to 5 seconds. FIG. 12B shows a bar chart comparing the storage and loss modulus at distances from 2 cm to 10 cm. FIGS. 13A-13D show results of EGF surface-coupling by V-CXL in a rodent corneal debridement animal model. A 2 mm corneal debridement was performed in three groups of two rodent eyes each (average corneal diameter=3 mm). Shown here are photographs of fluorescein-stained rodent eyes at 24 hours: EGF encapsulated within a collagen gel using riboflavin and blue light exposure for 5 seconds (FIG. 13A), EGF coupling directly to corneal stroma using riboflavin and blue light exposure for 5 seconds (FIG. 13B), topical EGF only (FIG. 13C), and no treatment (FIG. 13D). Slit lamp photos of the wounded and treated eyes are shown on the left, and fluorescein staining of the same eyes are shown on the right. In this limited cohort of rodents, EGF encapsulation within a collagen gel and direct EGF binding to corneal stroma using riboflavin and 5 seconds of blue light exposure shows greater wound area reduction at 24 hours than topical EGF or no treatment. FIG. 13E shows the average relative wound area intensity by fluorescein staining in arbitrary units quantified from the data shown in FIGS. 13A-D.

FIG. 14 shows an ocular treatment system using visible blue light to anchor growth factors to corneal wounds and enhance healing through rapid re-epithelialization and corneal nerve regeneration. The ocular treatment system comprises a drug-device combination to enhance wound healing composed of (1) an aqueous solution of recombinant growth factors mixed with an FDA-approved photosensitizer (e.g., riboflavin), and (2) a blue LED (˜458 nm) light source.

FIG. 15 shows a schematic of visible light-based riboflavin crosslinking (V-CXL) of growth factors to the cornea. A solution of growth factors and riboflavin are applied to bare corneal stroma (top). Visible light (˜458 nm) is applied (middle), which immobilizes the growth factors to the wound bed (bottom). Over time, the growth factors are slowly release as the surrounding matrix is turned over.

FIG. 16 shows a schematic of a visible light-based riboflavin crosslinking using of a gel that is formed in situ and encapsulates growth factors on the surface of damaged tissue. In one example, collagen solution is mixed with riboflavin and a growth factor and is exposed to blue light on the surface of a wounded cornea. This leads to gelation of the collagen around the growth factor and adherence of the gel to the corneal stroma. This leads to a sustained release of the growth factors as the applied matrix is broken down and turned over.

FIG. 17 shows a schematic of a visible light-based riboflavin crosslinking using of a gel that is formed in situ and encapsulates cells on the surface of damaged tissue. In one example, collagen solution is mixed with riboflavin and hMSCs and is exposed to blue light on the surface of a wounded cornea. This leads to gelation of the collagen around the hMSCs and adherence of the gel to the corneal stroma, creating a “living reservoir” of secreted therapeutic factors from the encapsulated hMSCs.

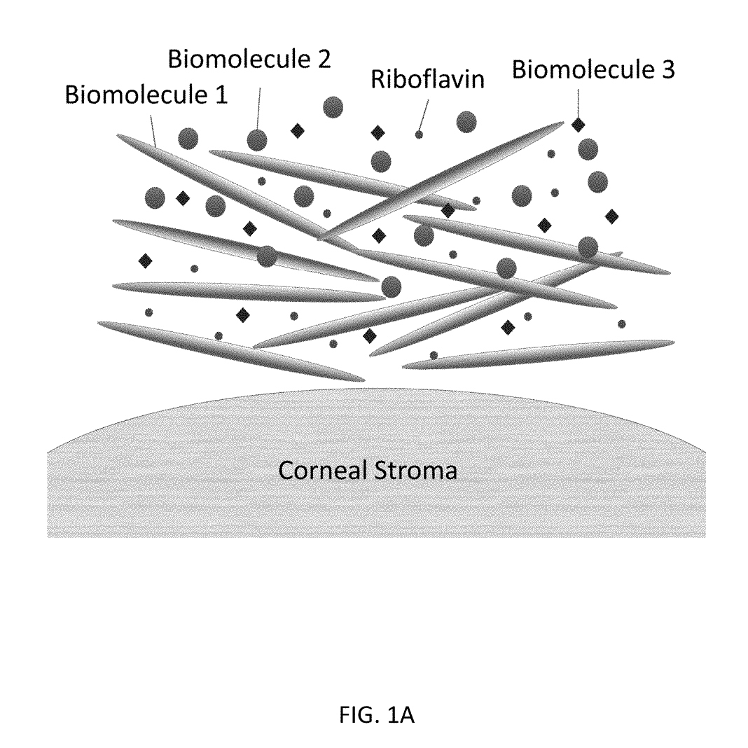

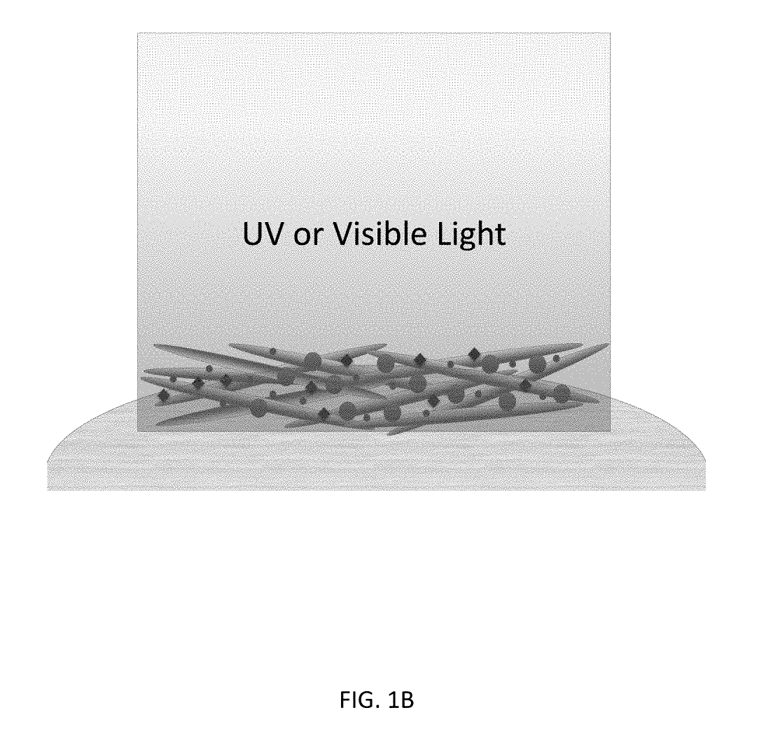

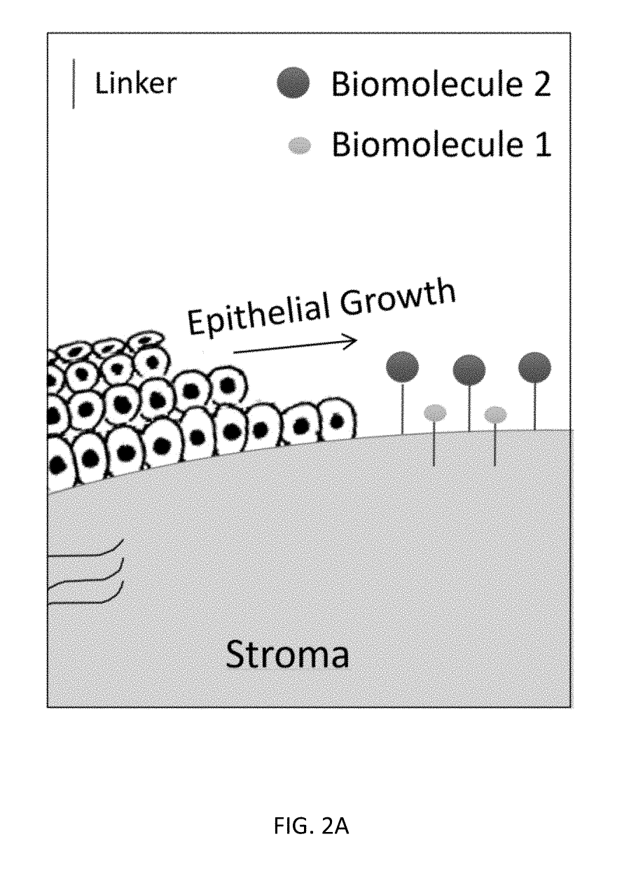

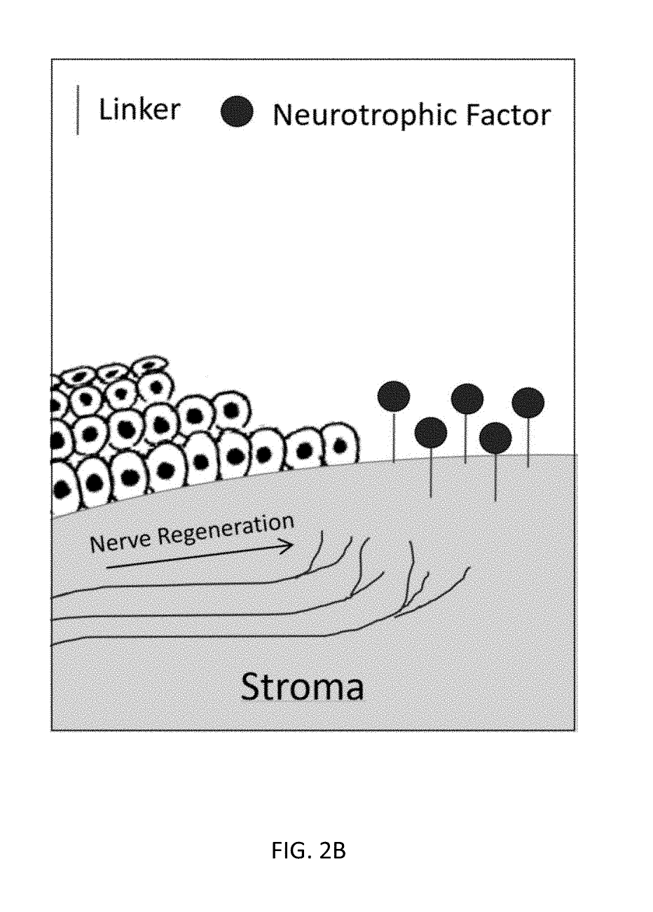

### DETAILED DESCRIPTION

The practice of the present invention will employ, unless otherwise indicated, conventional methods of medicine, pharmacology, chemistry, biochemistry, molecular biology and recombinant DNA techniques, within the skill of the art. Such techniques are explained fully in the literature. See, e.g. S. S. Wong and D. M. Jameson Chemistry of Protein and Nucleic Acid Cross-Linking and Conjugation (CRC Press, 2^{nd} edition, 2011); G. T. Hermanson Bioconjugate Techniques (Academic Press, 3^{rd} edition, 2013); B. Bowling Kanski's Clinical Ophthalmology: A Systematic Approach, 8e (Saunders Ltd., 8^{th} edition, 2015); A. L. Lehninger, Biochemistry (Worth Publishers, Inc., current addition); Sambrook et al., Molecular Cloning: A Laboratory Manual (3^{rd} Edition, 2001); and Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a biomolecule” includes two or more biomolecules, and the like.

The term “about,” particularly in reference to a given quantity, is meant to encompass deviations of plus or minus five percent.

A “wound” is a break or discontinuity in the structure of an organ or tissue, including epithelium, connective tissue, and muscle tissue. Examples of wounds include, but are not limited to, skin wounds, burns, bruises, ulcers, bedsores, grazes, tears, cuts, punctures, perforations, corneal abrasions and disruptions, corneal damage caused by neurotrophic keratopathy and exposure keratopathy, and neurotrophic recurrent corneal erosions. A wound may include tissue damage produced by a surgical procedure, trauma, or disease.

“Topical” application refers to non-systemic local administration of an active ingredient (e.g., biomolecule or photosensitizer) to a surface or subsurface of damaged tissue or a wound.

The term “subject” includes both vertebrates and invertebrates, including, without limitation, mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like.

“Treatment” of a subject or “treating” a subject for a disease or condition herein means reducing or alleviating clinical symptoms of the disease or condition, including tissue damage or loss, nerve damage, or impaired or slow wound-healing.

By “therapeutically effective dose or amount” of a therapeutic factor is intended an amount that, when administered in combination with a photosensitizer as described herein, brings about a positive therapeutic response in a subject having tissue damage or loss, such as an amount that improves wound healing or nerve regeneration. A therapeutically effective amount of a therapeutic factor may, for example, accelerate healing of damaged tissue, increase thickness of an epithelial layer of the damaged tissue, increase rate of epithelialization at the site of damaged tissue, shorten the time required for wound closure, or promote nerve regeneration in the damaged tissue. The exact amount required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

The terms “peptide,” “oligopeptide,” and “polypeptide” refer to any compound comprising naturally occurring or synthetic amino acid polymers or amino acid-like molecules including but not limited to compounds comprising amino and/or imino molecules. No particular size is implied by use of the terms “peptide,” “oligopeptide” or “polypeptide” and these terms are used interchangeably. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, linearly-linked peptides), cyclized, branched molecules and the like, are included within the definition. The terms also include molecules comprising one or more peptoids (e.g., N-substituted glycine residues) and other synthetic amino acids or peptides. (See, e.g., U.S. Pat. Nos. 5,831,005; 5,877,278; and 5,977,301; Nguyen et al. (2000) Chem Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, polypeptides useful in this invention can have a maximum length suitable for the intended application. Preferably, the polypeptide is between about 40 and 300 residues in length. Generally, one skilled in art can easily select the maximum length in view of the teachings herein. Further, peptides and polypeptides, as described herein, for example synthetic peptides, may include additional molecules such as labels or other chemical moieties. Such moieties may further enhance stimulation of epithelial cell proliferation and/or wound healing, and/or nerve regeneration, and/or biomolecule stability or delivery.

Thus, references to polypeptides or peptides also include derivatives of the amino acid sequences of the invention including one or more non-naturally occurring amino acids. A first polypeptide or peptide is “derived from” a second polypeptide or peptide if it is (i) encoded by a first polynucleotide derived from a second polynucleotide encoding the second polypeptide or peptide, or (ii) displays sequence identity to the second polypeptide or peptide as described herein. Sequence (or percent) identity can be determined as described below. Preferably, derivatives exhibit at least about 50% percent identity, more preferably at least about 80%, and even more preferably between about 85% and 99% (or any value therebetween) to the sequence from which they were derived. Such derivatives can include postexpression modifications of the polypeptide or peptide, for example, glycosylation, acetylation, phosphorylation, and the like.

Amino acid derivatives can also include modifications to the native sequence, such as deletions, additions and substitutions (generally conservative in nature), so long as the polypeptide or peptide maintains the desired activity (e.g., promote epitheilial cell proliferation and wound healing). These modifications may be deliberate, as through site-directed mutagenesis, or may be accidental, such as through mutations of hosts that produce the proteins or errors due to PCR amplification. Furthermore, modifications may be made that have one or more of the following effects: increasing specificity or efficacy of biomolecule, enhancing epithelial cell proliferation, wound healing, and/or nerve regeneration, and facilitating cell processing.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

### II. MODES FOR CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The wound healing response is often limited or impaired in patients with diabetic ulcers, burns, chemical exposure injuries, traumatic injuries, surgical wounds, neurotrophic keratopathy, or nerve damage. Thus, better ways are needed to stimulate a regenerative response in order to foster wound healing, restore anatomy and, in turn, tissue functions such as epithelial barrier effects and neural transmission.

The invention is based on the discovery that crosslinking therapeutic biomolecules directly onto damaged tissue (either on the surface or beneath the surface, e.g. in stromal or subcutaneous tissue) through the application of light in the presence of a photosensitizer improves wound healing (See Examples). Although topically applied growth factors have shown promise in treating chronic wounds, ligands can be depleted from the environment by endocytosis of growth factor-receptor complexes (Lee et al. (2011) J R Soc Interface 8(55):153-170, Schultz et al. (2009) Wound Repair and Regeneration 17(2):153-162). Chronic wounds also exhibit increased levels of enzymes like matrix metalloproteases (MMPs) that rapidly break down provisional matrices laid down by local fibroblasts (Schultz et al., supra).

Photochemical crosslinking has the potential to overcome these problems by immobilizing ligands and increasing the resistance of the extracellular matrix to enzymatic degradation (Spoerl et al. (2004) Curr Eye Res 29(1):35-40).

In order to further an understanding of the invention, a more detailed discussion is provided below regarding methods of crosslinking therapeutic factors directly onto tissues to promote wound healing.

### A. Light Activated Crosslinking of Therapeutic Factors onto Tissue

Therapeutic factors that can be used in the practice of the invention include any biomolecule, drug, or cell, which when administered in combination with a photosensitizer as described herein, promotes tissue repair or regeneration. Therapeutic factors may, for example, accelerate healing, increase thickness of an epithelial layer, increase the rate of epithelialization, shorten the time required for wound closure, or promote nerve regeneration in damaged tissue. In certain types of wounds, one or more of the following biomolecules may be needed for healing: a scaffold for cell adhesion (e.g., a functional extracellular matrix), a stimulus for cell proliferation (e.g., growth factors), nerve signaling (e.g., neuropeptides), and axon guidance proteins for nerve regeneration. Exemplary therapeutic factors that can be used include growth factors, such as epidermal growth factor (EGF), nerve growth factor (NGF), vascular endothelial growth factor (VEGF), and insulin-like growth factor (IGF); neuropeptides, such as substance P (SP) and calcitonin gene-related peptide; extracellular matrix proteins, such as fibronectin, collagen, laminin, and fibrin; axon guidance proteins, such as netrins (e.g., netrin-1), ephrins, and cell adhesion molecules; and other biomolecules that play various roles in tissue regeneration, such as beta-thymosins (e.g., thymosin beta-4). Other types of molecules or biomolecules may also be used, such as anti-vascular endothelial growth factor (anti-VEGF) therapeutic agents to prevent vascularization, leakage, or growth. Tethering anti-VEGF therapeutic agents (e.g., bevacizumab and ranibizumab) to tissues may be useful, for example, in the treatment of certain cancers or proliferative conditions, including wet macular degeneration or diabetic retinopathy. Additionally, therapeutic factors may include antibiotic agents, antifibrotic agents, anti-inflammatory agents, chemotherapeutic (anti-oncologic) agents, anti-angiogenic agents, or anti-thrombotic agents, and pro-thrombotic agents.

Therapeutic factors may be attached to tissue in a number of ways. In one embodiment, therapeutic factors are crosslinked onto a wounded tissue surface through photochemical means. A photosensitizer is applied to the tissue followed by exposure to non-visible or visible light (e.g., UV, white light, or blue visible light) at a suitable wavelength to initiate the crosslinking reaction resulting in the formation of covalent bonds with surrounding biomolecules or macromolecules of the tissue. Exemplary photosensitizers include riboflavin, rose bengal, eosin, and methylene blue, which upon exposure to light, produce reactive singlet oxygen and free radicals that generate covalent bonds between adjacent segments of macromolecules that contain carbonyl functional groups. The appropriate wavelength for initiation of photochemical reactions depends on the photosensitizer that is used. For example, riboflavin absorbs UV light (360-370 nm) and blue visible light (about 458 nm), rose bengal and eosin both absorb green light (480-550 nm), and methylene blue absorbs visible light in the yellow to red range (550-700 nm). Additionally, molecules containing photo-activatable reactive chemical groups such as aryl azides and diazirines can be used as photosensitizers. For example, exposure of azidobenzamido groups to UV light (250-320 nm) generates aromatic nitrenes, which can insert into a variety of covalent bonds. Exposure of diazirines to UV light (330-370 nm) generates reactive carbene intermediates, which can form covalent bonds through addition reactions with amino acid side chains or the peptide backbone of proteins. For a description of photosensitizers and photocrosslinking techniques, see, e.g., DeRosa et al. (2002) Coordination Chemistry Reviews 233-234:351-371, Kamaev et al. (2012) Invest Ophthalmol Vis Sci 53(4):2360-2367, Mastropasqua et al. (2015) Eye Vis 2:19, Lombardo et al. (2015) J Cataract Refract Surg. 41(2):446-459, Omobono et al. (2015) J Biomed Mater Res A. 103(4):1332-1338, Cherfan et al. (2013) Invest Ophthalmol Vis Sci. 54(5):3426-3433, Liu et al. (1999) Methods Mol Biol. 118:35-47, S. S. Wong and D. M. Jameson Chemistry of Protein and Nucleic Acid Cross-Linking and Conjugation (CRC Press, 2^{nd} edition, 2011), G. T. Hermanson Bioconjugate Techniques (Academic Press, 3^{rd} edition, 2013); herein incorporated by reference in their entireties).

Multiple therapeutic factors can be mixed with a photosensitizer and crosslinked simultaneously. Alternatively, different photosensitizers or photochemical linking strategies can be used with different therapeutic factors or subsets of therapeutic factors, and separate crosslinking reactions can be carried out sequentially in a number of discrete steps. For instance, one photosensitizer can be used with one therapeutic factor or subset of therapeutic factors, and another photosensitizer can be used with another therapeutic factors or subset of therapeutic factors.

In addition, a photosensitizer may include a second crosslinking moiety, which is not light-activatable at the wavelength used to initiate photochemical crosslinking. The use of an additional reactive group allows therapeutic factors to be linked to the photosensitizer via the non-light-activatable crosslinking moiety to produce a light-activatable bioconjugate of the therapeutic factors. Such bioconjugates can also be used to crosslink biomolecules directly onto tissue upon exposure to light.

Such heterobifunctional crosslinking agents may include, for example, dimethyl suberimidate, N-hydroxysuccinimide, or formaldehyde. In addition, carboxyl-reactive chemical groups such as diazomethane, diazoacetyl, and carbodiimide can be included for crosslinking carboxylic acids to primary amines. In particular, the carbodiimide compounds, 1-ethyl-3-(-3-dimethylaminopropyl) carbodiimide hydrochloride (EDC) and N′,N′-dicyclohexyl carbodiimide (DCC) can be used for conjugation with carboxylic acids. In order to improve the efficiency of crosslinking reactions, N-hydroxysuccinimide (NETS) or a water-soluble analog (e.g., Sulfo-NHS) may be used in combination with a carbodiimide compound. The carbodiimide compound (e.g., EDC or DCC) couples NHS to carboxyl groups to form an NHS ester intermediate, which readily reacts with primary amines at physiological pH. For a description of various crosslinking agents and bioconjugation techniques, see, e.g., G. T. Hermanson Bioconjugate Techniques (Academic Press, 3^{rd} edition, 2013), herein incorporated by reference in its entirety.

Therapeutic factors and photosensitizers are applied to damaged tissue at a surface or a subsurface. For example, one or more therapeutic factors and photosensitizers may be applied at the surface of tissue (e.g., to promote wound closure) or beneath the surface (e.g. in stromal or subcutaneous tissue), or at the location of a damaged nerve (e.g., to promote nerve regeneration). In addition, damaged tissue may be prepared prior to treatment by exfoliation or debridement of fibrotic or necrotic areas.

In certain embodiments, one or more therapeutic factors are applied to the damaged tissue in a pattern, tracks, or a gradient. For example, a gradient of growth factors or axon guidance factors can be used, e.g., to guide cell migration or nerve regeneration. A gradient can be produced, for example, by varying light intensity, the length of light exposure, or the concentration of biomolecules along the damaged tissue.

Upon exposure to light, a photosensitizer reacts with surrounding molecules, including the therapeutic factors and the proteins of the tissue, resulting in crosslinking (i.e., formation of direct bonds) between the therapeutic factors and the tissue and the therapeutic factors among one another. Biomolecules may include more than one functional group that can be crosslinked to allow formation of bonds among multiple biomolecules and a tissue surface or subsurface.

### B. Applications

The methods of the invention can be applied to any number of medical applications where tissue regeneration or improved wound healing is needed. Any condition where healing is impaired may benefit from such treatment such as, but not limited to a diabetic ulcer, a neurotrophic ulcer, a burn, a chemical injury, a skin injury, a nerve injury, or an eye injury.

For example, corneal damage, particularly persistent corneal epithelial defects can be treated by photochemically binding biomolecular assemblies directly to damaged stroma. In particular, damage to corneal tissue, such as caused by neurotrophic keratopathy, recurrent corneal erosion, a corneal ulcer, exposure keratopathy, or physical trauma may be treated in this manner. The corneal surface can be prepared by optionally debriding the edges of an epithelial defect and its base, followed by application of a photosensitizer and one or more therapeutic factors to the surface using sterile week-cells, and then exposing the surface to UV or visible light, depending on the selected photosensitizer, to initiate photocrosslinking. For UV crosslinking, an optional contact lens (non-UV absorbing) can be placed at the time of a UV exposure to limit the crosslinking reaction to the corneal surface. An optional bandage contact lens can also be placed after the reaction.

In another example, the methods are applied to skin wound healing. For example, a diabetic foot ulcer is treated by first debriding fibrotic or necrotic areas, followed by application of one or more therapeutic factors and a photosensitizer, and exposure to light,

This technology also has applications in cellular therapy, including allogeneic cell therapy, autologous cell therapy, and stem cell therapy. For example, the technology can be used to create a niche for cells at the time of transplantation. In addition, cells can be encapsulated in a biomolecular assembly, created by the methods of the invention, to provide a scaffold for proliferation, growth, and differentiation. For example, this technology can be used in applications where stem cells are needed, such as in corneal limbal stem cell deficiency.

This method can also be used to create implantable tissue substitutes made from explanted tissue, cultured cells, encapsulated cells within matrices, bio-artificial polymers, proteins and/or peptides or some combination thereof. A physical matrix or membrane can be configured to act as a wound dressing or overlay, similar to an amniotic membrane, but comprised of a specific and known formulation of biomolecules.

### C. Pharmaceutical Compositions

Therapeutic factors and photosensitizers can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations. Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of therapeutic factors and/or photosensitizers (e.g., when contained in a drug delivery system) in a composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for topical, subcutaneous, or localized delivery.

The pharmaceutical preparations herein can also be housed in a syringe, an implantation device, a microneedle injection system, or the like, depending upon the intended mode of delivery and use. Preferably, the compositions comprising therapeutic factors and/or photosensitizers, prepared as described herein, are in unit dosage form, meaning an amount of a conjugate or composition of the invention appropriate for a single dose, in a premeasured or pre-packaged form.

The compositions herein may optionally include one or more additional agents, such as other drugs for treating a wound or tissue damage, or other medications used to treat a subject for a condition or disease. Compounded preparations may be used including therapeutic factors and/or photosensitizers and one or more other drugs for treating a wound or tissue damage, such as, but not limited to, analgesic agents, anesthetic agents, antibiotics, anti-inflammatory agents, or other agents that promote wound healing. Alternatively, such agents can be contained in a separate composition from the composition comprising biomolecules and co-administered concurrently, before, or after the composition comprising biomolecules.

### D. Administration

At least one therapeutically effective cycle of treatment with at least one therapeutic factor in combination with a photosensitizer will be administered to a subject in need of tissue regeneration or repair. By “therapeutically effective dose or amount” of a therapeutic factor is intended an amount that, when administered in combination with a photosensitizer as described herein, brings about a positive therapeutic response in a subject having tissue damage or loss, such as an amount that improves wound healing or nerve regeneration. A therapeutically effective amount of a therapeutic factors may, for example, accelerate healing of damaged tissue, increase thickness of an epithelial layer of the damaged tissue, increase rate of epithelialization at the site of damaged tissue, shorten the time required for wound closure, or promote nerve regeneration in the damaged tissue. Additionally, an “effective amount” of a photosensitizer is an amount sufficient for photochemically crosslinking biomolecules directly onto tissue.

In certain embodiments, multiple therapeutically effective doses of compositions comprising one or more therapeutic factors and/or photosensitizers and/or one or more other therapeutic agents, such as other drugs or agents for treating a wound or damaged tissue, or other medications will be administered. The compositions of the present invention are typically, although not necessarily, administered topically, via injection (subcutaneously or intramuscularly), by infusion, or locally. Additional modes of administration are also contemplated, such as transdermal, intradermal, and so forth.

The preparations according to the invention are also suitable for local treatment. Compositions comprising one or more biomolecules and/or photosensitizers may be administered directly on the surface of a wound, adjacent to a wound, or beneath the surface of a wound (e.g. in stromal or subcutaneous tissue). Additionally, compositions may be applied at the location of a damaged nerve (e.g., to promote nerve regeneration). For example, a composition may be administered by spraying the composition on a wound, or as drops or a topical paste. Therapeutic factors and photosensitizers may also be added to wound dressings. A wound dressing may comprise, for example, a gel, a viscoelastic solution, putty, a physical matrix or a membrane. The particular preparation and appropriate method of administration are chosen to effect photochemical-coupling of therapeutic factors at the site in need of tissue regeneration or repair.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising therapeutic factors, photosensitizers, and other agents may be administered using the same or different modes of administration in accordance with any medically acceptable method known in the art.

In another embodiment, the pharmaceutical compositions comprising therapeutic factors, photosensitizers, and/or other agents are administered prophylactically. Such prophylactic uses will be of particular value for subjects who suffer from a condition which impairs or slows down the healing of a wound or causes tissue damage or prior to a procedure that will cause tissue damage.

In another embodiment of the invention, the pharmaceutical compositions comprising biomolecules and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising therapeutic factors (e.g. biomolecule-photosensitizer conjugate) as provided herein to a patient suffering from a condition that is responsive to treatment with biomolecules contained in the conjugate or composition. The method comprises administering, via any of the herein described modes, a therapeutically effective amount of the conjugate or drug delivery system, preferably provided as part of a pharmaceutical composition. In one embodiment, hyaluronic acid is conjugated in one batch with thiols groups and conjugated in a second batch with acrylate or methacrylate groups. When mixed together and exposed to UV or blue light in the presence of riboflavin, a so-called photoinitiated “thiol-ene” or “photo-click” reaction takes place that rapidly forms a hyaluronic acid gel. This gel can be used alone or to encapsulate other biomolecules such as growth factors (with or without thiol or acrylate/methacrylate functionality) on wounds to promote healing as described herein.

The actual dose of therapeutic factors in combination with a photosensitizer to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case. The amount of therapeutic factors administered will depend on the potency of particular therapeutic factors and the magnitude of its effect on tissue regeneration and repair (e.g., wound epithelialization and healing, nerve regeneration) and the route of administration.

Therapeutic factors, prepared as described herein (again, preferably provided as part of a pharmaceutical preparation), can be administered alone or in combination with one or more other therapeutic agents for treating a wound or tissue damage, such as, but not limited to, analgesic agents, anesthetic agents, antibiotics, anti-inflammatory agents, or other agents that promote wound healing, or other medications used to treat a particular condition or disease according to a variety of dosing schedules depending on the judgment of the clinician, needs of the patient, and so forth. Therapeutic factors, drugs, or other agents can be trapped within a gel either through physical entanglements or via covalent bonds that are either non-specific or specific. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day. In some cases, only a single administration will be needed.

Therapeutic factors can be administered prior to, concurrent with, or subsequent to other agents. If provided at the same time as other agents, therapeutic factors can be provided in the same or in a different composition. Thus, therapeutic factors and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising therapeutic factors and a dose of a pharmaceutical composition comprising at least one other agent, such as another drug for treating a wound or damaged tissue, which in combination comprise a therapeutically effective dose, according to a particular dosing regimen. Similarly, therapeutic factors and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

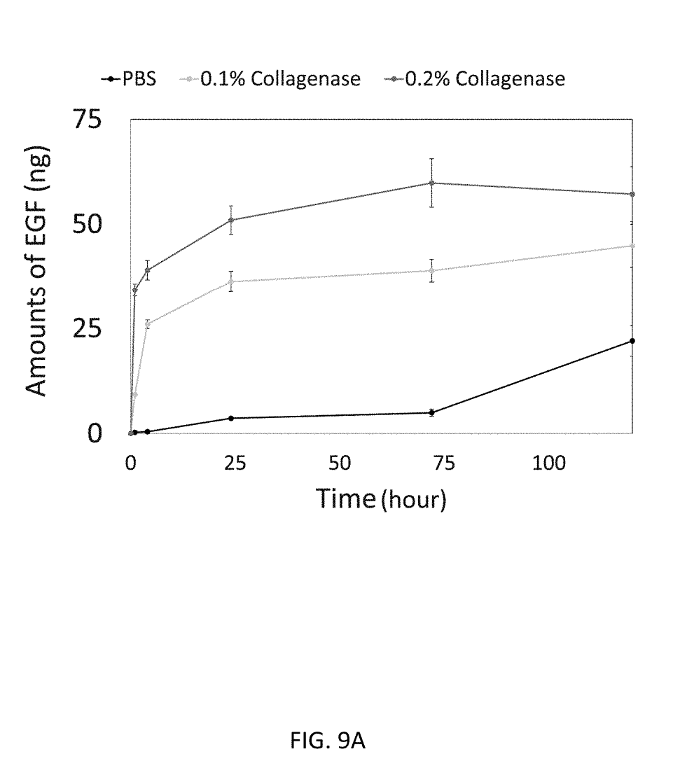

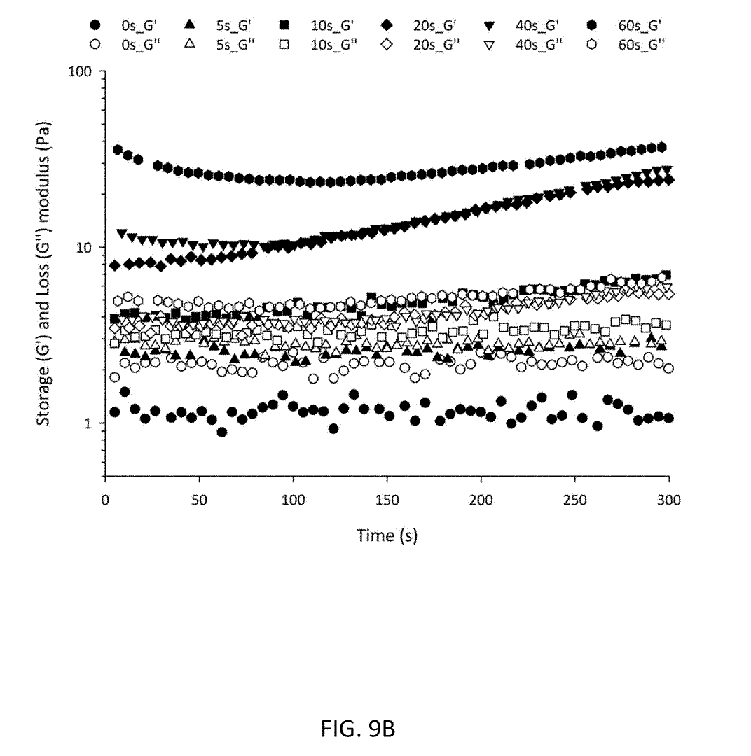

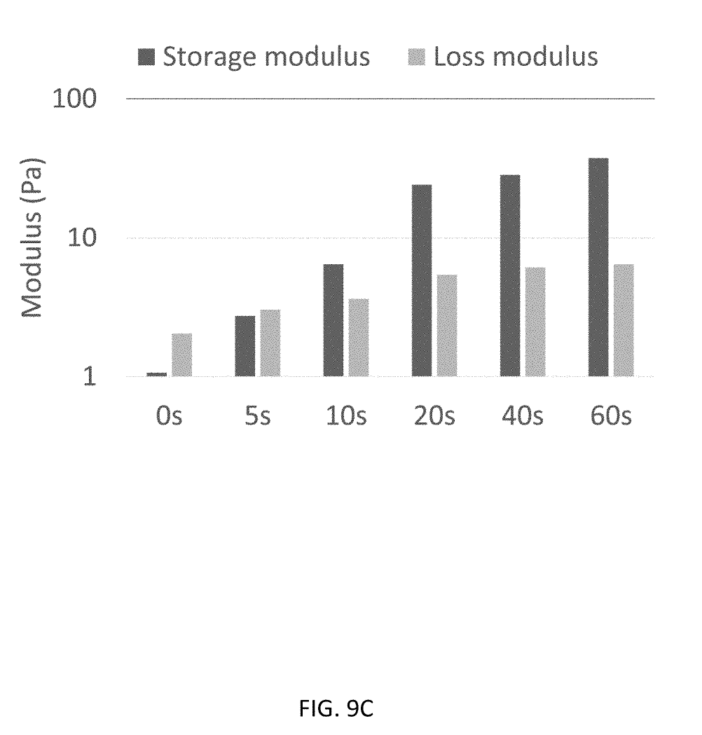

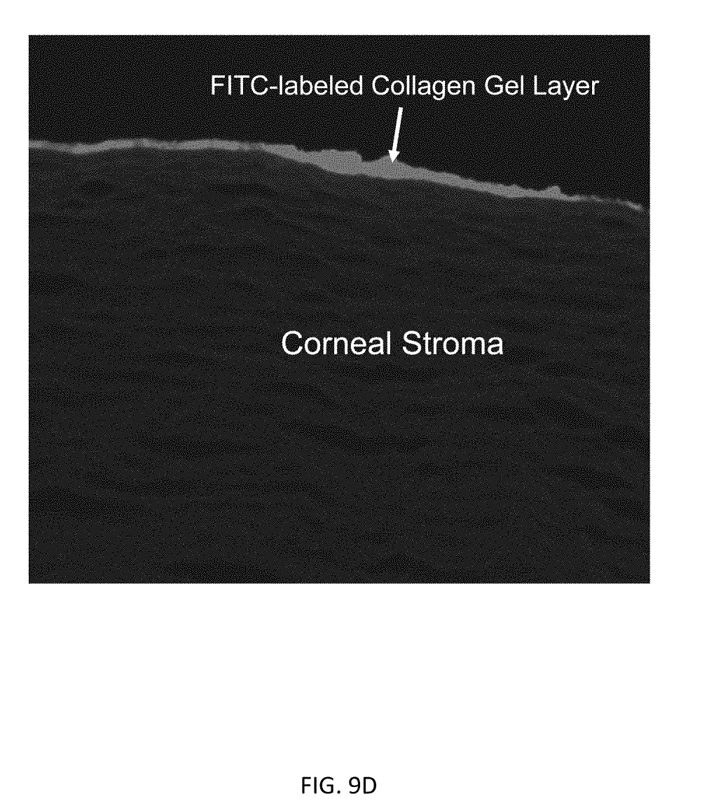

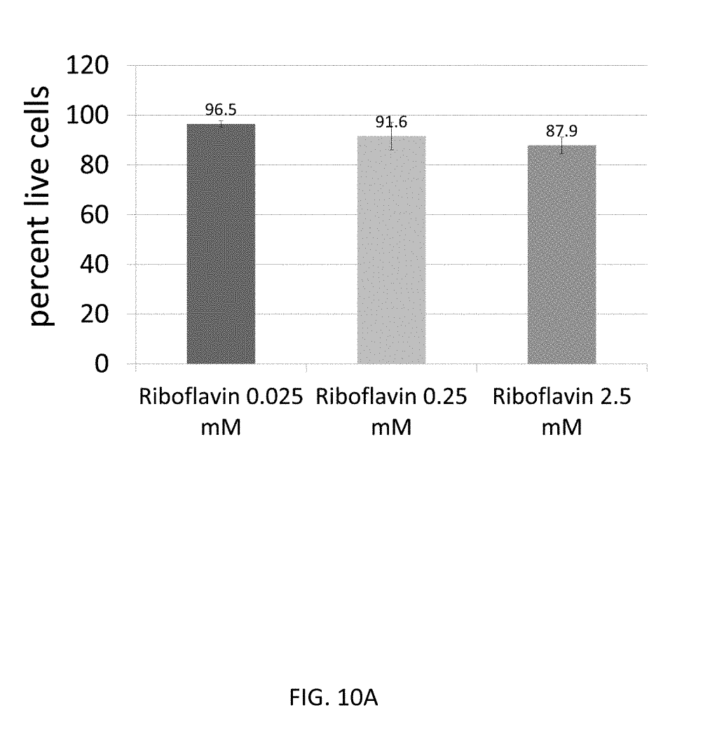

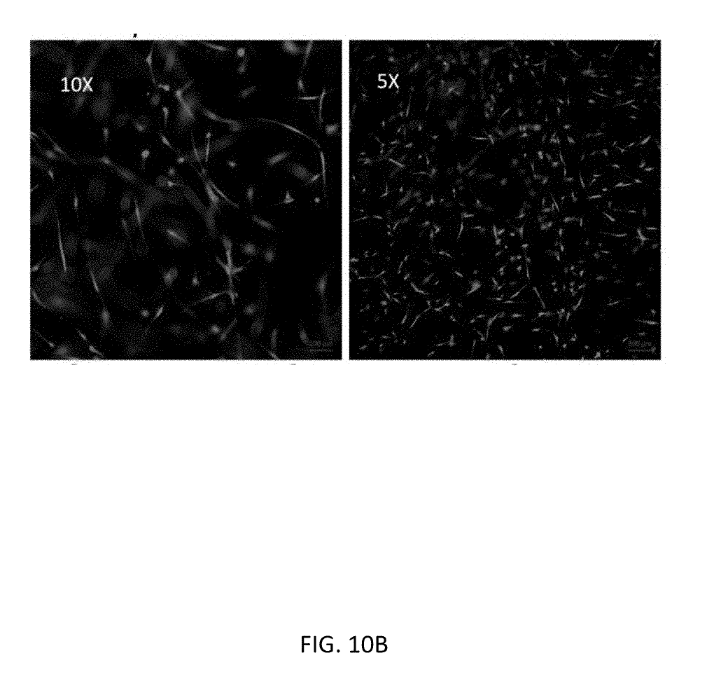

A major clinical challenge is the delivery of cells, particularly stem cells, to an diseased or damaged area of the body. Currently, the stem cells are typically injected in suspension to an area without a specific or effective means to have them target and adhere to a particular location. Providing a immobilizing matrix and microenvironment in which to settle and grow while remaining immobilized to that are would be an advancement in the art. Cells can be encapsulated within a biomolecular gel according to the present invention. As shown in FIG. 10, hMSCs were encapsulated within collagen gels by mixing a 5 mg/mL collagen solution with riboflavin at varying concentrations and exposed to blue light for 20 seconds, showing up to 99% viability at one week in the case of the 0.025 mM riboflavin concentrations. Using this method, hMSCs and other cell types can be encapsulated and positioned to a specific location in the body. For instance, a collagen gel encapsulating hMSCs can be formed and adhered in situ using blue light and riboflavin on the surface of a corneal wound, creating a “living reservoir” of therapeutic factors that are secreted by the immobilized hMSCs. The formed gel adheres to the target tissue due to crosslinks formed between the exogenously applied collagen and the collagen on the wound bed or target tissue which is shown by example in FIG. 9D.

In another embodiment, a different biomolecule can be used as the encapsulating matrix such as hyaluronic acid, using thiolated and methacrylated hyaluronic acid in the presence of riboflavin and blue light to create a gel formed by “photo-click” thiol-ene chemistry. In yet another embodiment, a different cell type such as epithelial cells, stromal, or endothelial cells can be encapsulated and injected and adhered on the outer surface, within a stromal defect or wound, or on the posterior surface of the cornea, respectively, in the case of damage or loss of that particularly cell type or tissue in that location. In further embodiments, cells can be encapsulated in situ with the present invention on or deep to the skin to help regenerate acute or chronic wounds, such as diabetic foot ulcers or traumatic injuries. Nerve cells or neurogenic stem cells may be encapsulated at sites of nerve injury to help foster regeneration of injured, degenerated, or diseased nerves. Finally, the encapsulation of cells can be done in combination with the encapsulation of growth factors or other therapeutic factors that help to facilitate the growth, differentiation of the encapsulated cells at the target site.

### E. Kits

The invention also provides kits comprising one or more containers holding compositions comprising therapeutic factors and/or photosensitizers, and optionally one or more other drugs for treating a wound or tissue damage, such as, but not limited to, analgesic agents, anesthetic agents, antibiotics, anti-inflammatory agents, or other agents that promote wound healing or tissue regeneration. Compositions can be in liquid form or can be lyophilized. Suitable containers for the compositions include, for example, bottles, vials, syringes, and tubes. Containers can be formed from a variety of materials, including glass or plastic. A container may have a sterile access port (for example, the container may be a vial having a stopper pierceable by a hypodermic injection needle). Additionally, the kit may contain a light source that produces light (e.g., UV or visible) at a wavelength capable of activating a photosensitizer included in the kit, a UV filter, a non-UV absorbing contact lens, or a topical applicator or dispenser.

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions describing methods for photochemically coupling biomolecules onto tissue as described herein. The package insert can be an unapproved draft package insert or can be a package insert approved by the Food and Drug Administration (FDA) or other regulatory body.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### General Overview of Method for Photochemically Crosslinking and Tethering Biomolecules to Tissues to Promote Healing

We have developed a method to target and bind one or more biomolecules directly onto damaged tissue (either on the surface or beneath the surface, e.g. in stromal or subcutaneous tissue) through the application of UV or visible (e.g. blue) light in the presence of a photosensitizer. Such biomolecules include but are not limited to growth factors (e.g., epidermal growth factor, nerve growth factor, vascular endothelial growth factor, and insulin-like growth factor), neuropeptides (e.g., Substance P), extracellular matrix proteins (e.g., fibronectin, collagen, laminin, or fibrin), inhibitory molecules (e.g. anti-vascular endothelial growth factor), beta thymosins (e.g., thymosin beta-4), and netrins (e.g., netrin-1) that may work synergistically together to promote tissue healing and/or regeneration. Any number or combination of biomolecules may potentially be used.

Irradiation with UV or visible (e.g. blue) light in the presence of a photosensitizer or other light-activated chemical functional group facilitates the crosslinking and surface-tethering of these trophic factors to create a biomolecular assembly that releases the trophic factors upon the proteolytic degradation of the formed matrix by in-growing cells. Matrikines are peptides and other trophic factors that are released upon degradation of a matrix; thus, the invention can be described as a “matrikine-like” biomolecular assembly. This technology can be combined with the transplantation of tissue and cells such as stem cells in order to provide pro-migratory tracks for adhesion, growth, and differentiation.

When solubilized trophic factors are not photochemically crosslinked or tethered to a tissue surface or subsurface, the local concentration of solubilized factors delivered topically is inefficient due to endocytosis, hydrolysis, proteolysis, or washing away (through reflex tears in the eye, for instance) of the unbound agents. Immobilization and concentration of topical agents at the surface of damaged tissue increases their residence time, as well as enabling the synergistic combination of multiple proteins to work together in a biomimetic, “matrikine-like” fashion. Formation of a matrikine-like assembly by using out methods provides spatial-temporal control over the regenerative process and has the advantage that it does not require frequent re-administration of the active ingredients in order to maintain a sufficiently high local concentration. The matrikine-like assembly mimics the action of naturally occurring matrikines such as laminin, which possess both cell adhesion and epidermal growth factor like domains, which are released upon degradation by adherent cells.

Also, this method can be used to create implantable tissue substitutes made from explanted tissue, cultured cells, encapsulated cells within matrices, bio-artificial polymers, proteins and/or peptides or some combination thereof. A gel, viscoelastic solution, putty, physical matrix or membrane can be configured from this invention to act as a wound dressing or overlay, similar to an amniotic membrane, but comprised of a specific and known formulation of biomolecules, and then crosslinked into place. Alternatively, the biomolecule formulation can change from one viscosity to another as a result of the crosslinking, or can change from a solution (or gel) to a formed (non-flowable) matrix as a result of the crosslinking. Furthermore, the biomolecule formulation may have one or more thickening agents or other additives that contribute to the viscosity of the solution applied to a surface prior to crosslinking. These additives may or may not be washed away after the crosslinking reaction.

### Key Steps of the Procedure

1. Selection of tissue in which to stimulate regenerative response

2. Selection of biomolecule or biomolecules, and/or cells to target to tissue surface or sub-surface; formulation of biomolecule solution in combination with a photosensitizer (e.g. riboflavin)

3. Optional preparation of tissue (e.g. Exfoliation, debridement or fibrotic or necrotic areas)

4. Topical application of biomolecule solution to tissue surface or interface (e.g. at location of damaged or severed nerve endings)

5. Exposure of tissue to UV light source (e.g. 365 nm) or visible light source (e.g. 458 nm)

6. Optional rinsing of exposed tissue (e.g. with sterile saline)

7. Optional topical application of other therapeutic agents

Step #4 may comprise the combination of all biomolecules in Step #2 simultaneously, or one or more of the biomolecules in Step #2 followed by Step #3-6, then repeating Steps 4-6 again for the second biomolecule, then again for the third biomolecule, etc. . . . In other words, biomolecules can be crosslinked sequentially rather than simultaneously. A photosensitizer such as riboflavin in the presence of UV or visible (e.g., blue light) irradiation facilitates intra- and inter-molecular crosslinks within tissues, such as between collagen fibrils, but also with any biomolecules in the vicinity.

### Key Components

Therapeutic biomolecules, a photosensitizer, and a UV or visible light source are the key components needed to carry out this procedure. Damaged tissue, in the presence of the biomolecules, photosensitizer, and UV or visible light, are decorated with the biomolecules through newly formed chemical linkages.

Optional components are a non-UV absorbing contact lens, UV filter, and topical applicators. UV/visible light exposure time, intensity, wavelength, fractionation, as well as, photosensitizer concentration/identity, can all be varied to achieve the desired effect. The bioactivity and degradability are influenced by these variables and can be optimized for each particular application.

The photosensitizer can be added at the same, before or after the application of the biomolecules. The biomolecules can be added in sequence, simultaneously, or some combination thereof. For instance, two biomolecules can be used first, followed by one biomolecule. The biomolecules can be immobilized on or within tissue in a gradient-like fashion; this is advantageous in that it is known in the art that wound healing can occur along gradients of growth factors. These gradients can be formed by differential exposure to UV/visible light intensity, and/or varying time of exposure and/or through differential concentration of biomolecules on or within the tissue (for instance, through diffusion, injection, or infusion).

The photosensitized or photoinitiator may be a separate molecule that catalyzes the reaction or participates in the reaction. Alternative photoactive substances that can be used include molecules containing phenyl azides. For instance, a molecule can contain a phenyl azide on one side and an N-hydroxysuccinimide on another side. The N-hydroxysuccinimide side can react with a biomolecule or biomolecules in a light-free reaction, leaving the light-sensitive phenyl azide still available. Upon exposure to light, the phenyl azide can then react with any surface, including the proteins on tissues, thus creating a direct bond between the molecule and the tissue. Any biomolecule with more than one functional group can be used in this way, including those with long spacer arms between the functional groups, or with multiple arms (e.g. multi-functional, such as dendrimers) that can enable bonds between multiple biomolecules and a surface.

In another variation, a physical membrane or matrix is performed as a gel or a sheet (in hydrated or dehydrated or partially dehydrated form) through the application of a chemical or photochemical reaction, then bonded to a tissue through a subsequent chemical or photochemical reaction. In yet another variation, combinations of biomolecules with other biomolecules, or hybrid gels combining biomolecules and synthetic polymers such as polyethylene glycol, polyvinyl alcohol, poly(lactic-co-glycolic acid) (PLGA), polycaprolactone, polyacrylic acid can be used as the encapsulating matrix. In one example, collagen and 4-arm PEG-N-hydroxysuccinimide was used to encapsulate hMSCs using riboflavin and blue light.

### How to Perform the Procedure

The site of poorly healing tissue damage such as a diabetic ulcer, neurotrophic ulcer, burn, chemical injury, or nerve injury is prepared under sterile conditions for a medical procedure, including the use of antiseptics such as povidone iodine or antibiotic solution to clean the surface. The surface is also optionally further prepared by exfoliation or debridement to provide fresh edges of viable tissue that can be stimulated for growth. The surface or area of damage is then exposed to the biomolecule solution of choice using one of a variety of means, such as drop-wise delivery (for example to a small area), direct contact with a soaked weck-cell sponge or large sponge-like material, or other means of topically applying the solution to the surface. This solution may contain the photosensitizer as well (e.g. riboflavin or Rose Bengal), or the photosensitizer may be applied either before or after the biomolecule solution. A light source (typically UV or visible light source) is then applied to the surface for a designated time period or period (e.g. in a pulsed or fractionated pattern) to cause crosslinking of the biomolecules and tethering of the formed matrix to the tissue surface. A second or third biomolecule, or further additional biomolecules can be cross-linked and tethered to the surface in the same way. Various wavelengths of light can be used, including UV light, blue light, green light, as well as broad spectrum lights including white light/visible light. Any number of photosensitizers can be used, including but not limited to riboflavin and rose bengal.

Alternatively, other light-activatable chemical functional groups can be used to create the linkages, including phenyl azide-bearing linkers. Heterocrosslinkers such as those that contain phenyl azides on one end and N-hydroxysuccinimide functionality on another can also be used to create crosslinks between biomolecules and biomolecules with moieties on a tissue surface. Photochemistry may or may not be used to form linkages between solubilized proteins prior to tethering (for instance, chemical bonds are formed first between different types of biomolecules, and may or may not be used to bond these pre-linked biomolecules and a tissue). Although photochemistry may be used for the biomolecule-to-tissue reaction, this linkage may also be facilitated by non-photochemical means (such as reactions catalyzed by temperature, enzymes, or chemicals). Regardless of the chemical details, the method involves the use of compounds to create linkages between biomolecules and the proteins inherent to a tissue surface or subsurface.

### Example 2

### Treatment of Neurotrophic Keratopathy by Photochemically Immobilizing Biomolecules Directly onto Corneal Tissue

We improve the therapeutic potential of trophic biomolecules by photochemically binding them to the cornea in order to restore the neuropeptide signaling that is deficient in NK. This approach is designed to overcome the limitations of topical delivery, which requires frequent administration and does not provide sustained concentrations of therapeutic agents at the corneal surface where they are needed. Photochemical immobilization of growth factors has long been an effective strategy for promoting the adhesion and proliferation of cells on polymeric scaffolds (Kapur et al. (2003 J Biomater Sci Polym Ed. 2003;14(4):383-394; Kruse et al. (1999) Ophthalmology 106(8):1504-1511; Chen et al. (2000) Br J Ophthalmol 84(8):826-833; Bonini et al. (2000) Ophthalmology 107(7):1347-1351; Suzuki et al. (2003) Prog Retin Eye Res 22(2):113-133). This approach has been used successfully to support both corneal and nerve cell lines in vitro. In other studies, we have photochemically coupled EGF onto collagen-coated polymer surfaces to encourage corneal epithelialization (Myung et al. (2008) Invest Ophthalmol Vis Sci 49 (E-Abstract 5729), as well as the axon-guidance protein Netrin-1 onto polymer fibers to direct the radial growth of neurons (Kador et al. (2014) Acta Biomaterialia 10(12):4939-4946). In independent work, NGF has been tethered to hydrogel scaffolds to foster neural regeneration (Kapur et al., supra).

Here we photochemically immobilize growth factors using riboflavin and light exposure directly onto corneal stroma for the purpose of accelerating corneal wound healing and promote nerve regeneration in NK. Long-term epithelial stability requires the restoration of trigeminal innervation to the cornea through application of NGF.

Explanted animal corneas are debrided and photochemically modified with EGF and/or NGF. The corneas are evaluated for their bioactivity using a conformation-specific antibody, and surface concentration using mass spectrometry after a systematic series of UV exposure conditions. These experiments are used to better understand the photochemical conditions that dictate the bioactivity and surface concentration of the immobilized growth factors.

Although many types of growth factors and matrix molecules work together to mediate wound healing (Suzuki et al., supra), we focused on the photochemical binding of two types of stimuli: EGF is a small (6.1 kDa) protein that is heat-stable, relatively resistant to proteolysis, with potential to enhance corneal wound healing in soluble form that has been extremely well-characterized (Pastor et al. (1992) Cornea 11(4):311-314). NGF is a soluble protein belonging to a family of neurotrophic factors that on its own has been shown to be a facilitator of nerve regeneration in clinical trial settings for a variety of neuropathic conditions, including NK (Aloe et al. (2012) J Transl Med 10:239).

A heterobifunctional crosslinker is used to bind these biomolecules to the collagen in corneal stroma. The linker contains an N-hydroxysuccinimide (NHS) functional group on one end that reacts with primary amines on proteins, and a phenyl azide group on the other end that rapidly forms covalent bonds with adjacent macromolecules upon exposure to UV light. Pre-reacting growth factors with this agent produces a stable, light-activatable bioconjugate that can be stored in aqueous solution and applied to the cornea at a later time. This “azide-active ester” photocrosslinking strategy was chosen for the following reasons. First, it provides a relatively specific linkage on biomolecules through the NETS-group reaction with free primary amines, which reduces the likelihood (relative to a non-specific linkage) of affecting the native conformation of the linked proteins and, in turn, their bioactivity. Second, I have used this technique in prior work (Myung et al. (2008) Invest. Ophthalmol. Vis. Sci. 49 (E-Abstract 5729) to bind bioactive EGF to collagen-coated surfaces (described below). Third, the use of UV light to chemically modify the cornea has been extensively clinically tested for over ten years around the world through UV/riboflavin crosslinking (CXL), which safely and effectively forms covalent bonds between adjacent collagen fibrils (Kamaev et al. (2012) Invest Ophthalmol Vis Sci 53(4):2360-2367). This method has the potential to benefit patients with not only NK but also non-neurotrophic recurrent corneal erosions and ulcers, exposure keratopathy, and non-ocular wounds such as diabetic and venous stasis ulcers.

### Example 3

### Testing on Rat Corneas

Mouse corneas were subject to 2 mm diameter circular corneal debridement and then treated with (1) a collagen gel solution containing 0.01 mg/ml of EGF, and 0.1 mg/ml riboflavin and exposed to blue light (˜458 nm) for 5 seconds, (2) an aqueous EGF solution containing 0.01 mg/ml EGF and 0.1 mg/ml riboflavin and exposed to blue light for 5 seconds, (3) topical application of an aqueous solution of EGF alone at a concentration of 0.01 mg/mL, or (4) no treatment. The rodents were then examined at 24 hours and the wound areas examined by fluorescein staining. The results are shown in FIG. 13A-13E. Wound areas were found to be smaller at the 24 hour time point for the crosslinked collagen gel with EGF and the directly crosslinked EGF than in those treated with topical EGF alone or no treatment, indicating that the use of riboflavin and blue light to immobilize growth factors at a wound surface through either an encapsulating gel or directly to the wound can promote accelerated wound healing.

### Example 4

### Alternative Crosslinking Approaches

There are several alternatives to azide-active ester crosslinking. For example, ethyl(dimethylaminopropyl) carbodiimide (EDC)/NHS chemistry has been used successfully to conjugate a photocrosslinkable side group to an axon-guidance protein (Netrin-1), which was tethered directly to fibronectin on electrospun fibers (Kador et al. (2014) Acta Biomaterialia 10(12):4939-4946; herein incorporated by reference). Another possibility is riboflavin, which has been extensively tested as a natural photosensitizer that non-specifically forms crosslinks between collagen fibrils (Dunn et al. (2010) Ann. N.Y. Acad. Sci. 1194(1):199-206; Ehlers et al. (2009) J. Refract. Surg. 25(9):S803-6; Cummings et al. (2013) Indian J Ophthalmol 61(8):425-427), though its non-specificity provides less control over the bioactivity of the growth factors. Another possibility is rose bengal, which has been shown by Cherfan et al. to also be a photosensitizer that has been used in a variety of tissue applications not limited to corneal crosslinking (Cherfan et al. (2013) Invest. Ophthalmol. Vis. Sci. 54(5):3426-3433; herein incorporated by reference). Other crosslinking chemistries are possible as well, in particular ones where degradation chemistries such as caprolactone or acrylate moieties are employed to improve the release characteristics of the bound proteins.

### Example 5

### Crosslinking Biomolecules in Patterns, Tracks, or Gradients

A patterned approach to surface coupling can be taken where biomolecules (e.g., EGF, SP, and NGF) are crosslinked to stroma in discrete patterns or tracks, or in a gradient fashion (i.e., where a UV filter or blue light filter that provides more fluence centrally than peripherally). This approach may help epithelial cells and corneal nerves to migrate more readily. Indeed, the extracellular matrix has been shown to control growth factor presentation in a temporal and spatial fashion by stimulating migration along gradients of growth factors (Lee et al. (2011) J R Soc Interface 8(55):153-170). Moreover, growth factor concentration gradients have been shown to be an effective way to direct the growth of neurons (Kador et al. (2014) Acta Biomaterialia 10(12):4939-4946; Kapur et al. (2003) J. Biomater. Sci. Polym. Ed. 14(4):383-394). Another potential application is the formation of stem cell migratory tracks in vivo.

### Example 6

### Riboflavin-Based Corneal Crosslinking with Visible Light

Although riboflavin is approved for use in combination with UV light (e.g., 365 nm), riboflavin can also induce crosslinking between proteins in the presence of visible light (˜458 nm) (Ibusuki et al. (2007) Tissue Eng. 13(8):1995-2001). In order to improve the safety profile of riboflavin-based corneal crosslinking (CXL) in treating neurotrophic corneas, we used visible light (blue) to photoactivate riboflavin. In this application, we refer to this process as visible light crosslinking (V-CXL).

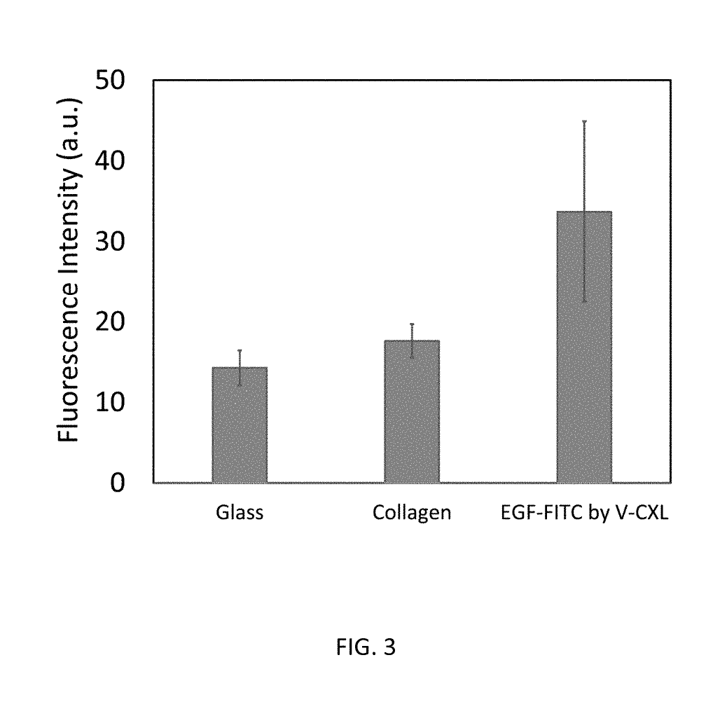

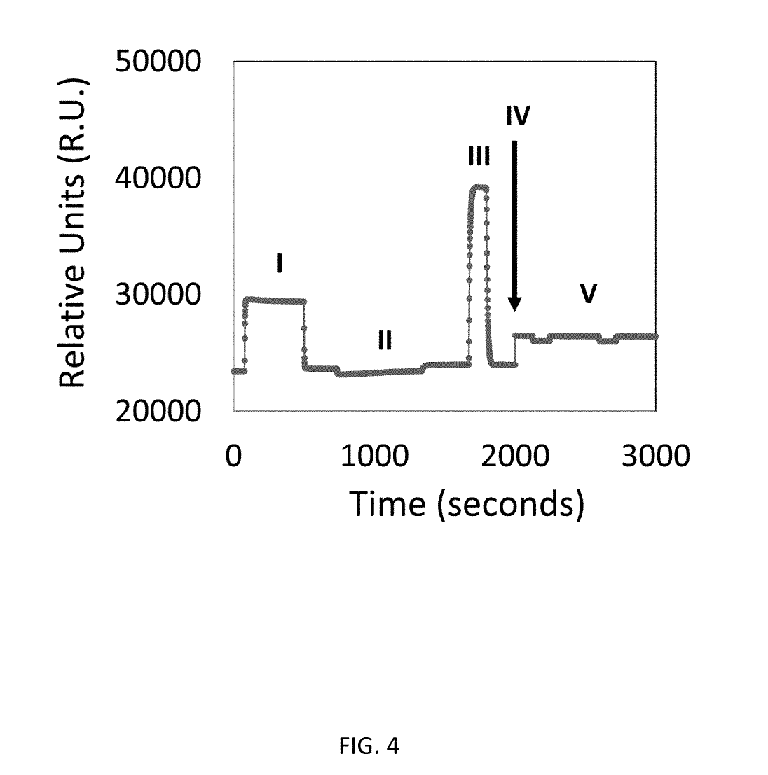

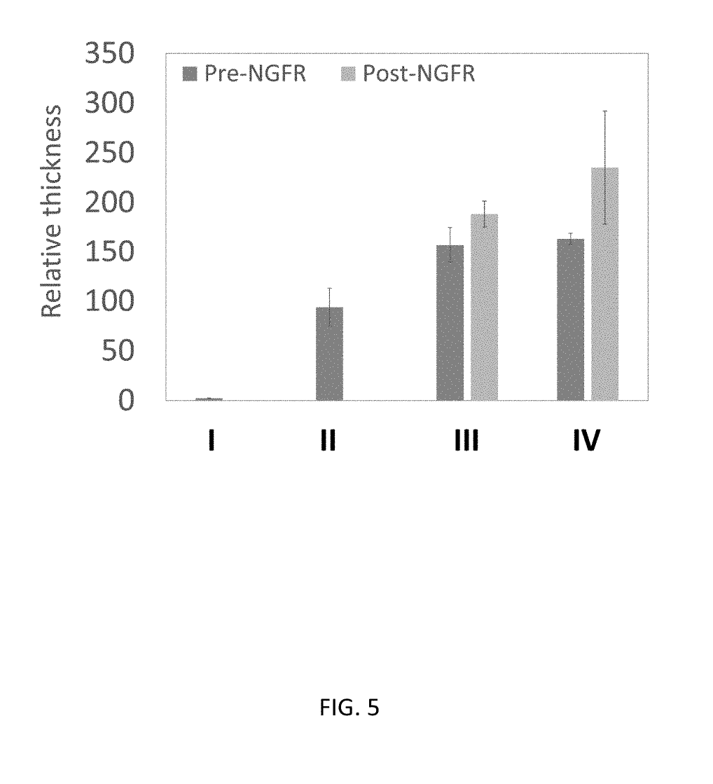

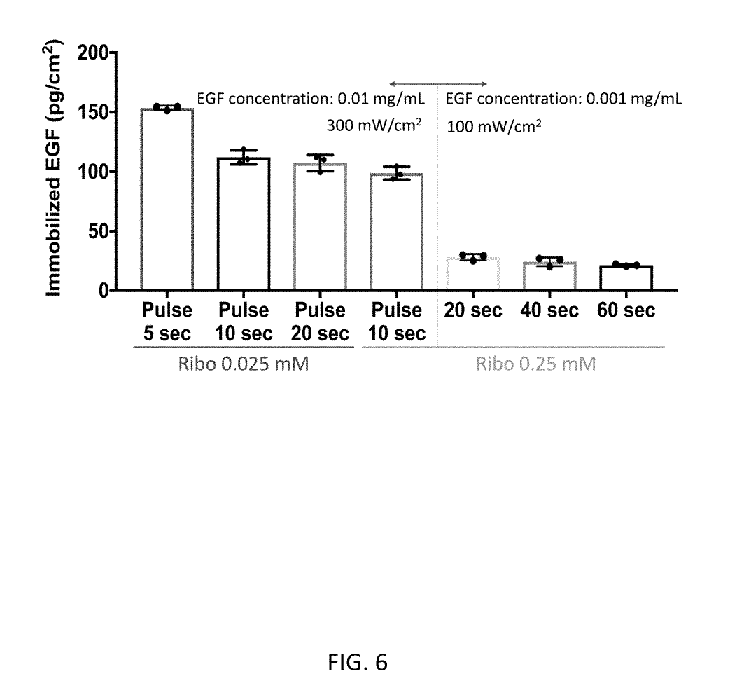

V-CXL binds growth factors directly to collagen. V-CXL was used to bind EGF and NGF growth factors directly to collagen. Fluorometry (FIG. 3), surface plasmon resonance (FIG. 4), ellipsometry (FIG. 5), and ELISA (FIG. 6) were used to monitor and compare the chemical coupling versus physical adsorption of NGF and EGF to collagen surfaces. In these experiments, collagen was first chemically immobilized to either gold, glass, or polystyrene surfaces (depending on the method being used), followed by V-CXL-mediated coupling of either EGF or NGF (or their fluorescein-isothiocyanate (FITC)-labeled conjugates) to the collagen coating alone or physical adsorption of growth factor to collagen from aqueous solution. Fluorometry (FIG. 3) showed an increase in fluorescence intensity (above background autofluorescence of the collagen/glass substrate) of FITC-labeled EGF coupled to collagen by V-CXL.

Exemplary surface plasmon resonance (SPR) results of NGF binding are shown in FIG. 4. Exposure of NGF to blue light in the presence of riboflavin led to a step-increase in the layer thickness of the collagen-coated gold wafer. Using ellipsometry (FIG. 5), the bioactivity of surface-coupled NGF was assessed by floating soluble NGF receptor over NGF-coupled surfaces, which showed that photochemical coupling of NGF to collagen yielded a higher amount of NGF receptor binding compared to physisorbed NGF.

FIG. 6 shows ELISA quantification of surface concentration (in pg/cm²) of EGF as a function of blue light exposure time using riboflavin-based CXL. Exposure times were varied by total time (2.5 sec to 60 sec) using either pulsed or constant exposure. The pulsed regimen involved 1 second on and on second off, so 5 seconds of pulsed exposure results in 2.5 sec of total blue light exposure. In these experiments, EGF of 0.01 mg/ml and 0.001 mg /ml were applied, with riboflavin at either 0.025 mM or 0.25 mM followed by blue light (˜458 nm) exposure at either 300 mW/cm² or 100 mW/cm² for 5, 10, 20, 40, or 60 seconds, with pulsed regimens of 5, 10, and 20 seconds. The results show that EGF surface binding for higher intensity blue light and lower concentration of riboflavin is generally optimized at shorter and pulsed exposure regimens.

### V-CXL Binds Growth Factors to Corneal Stroma

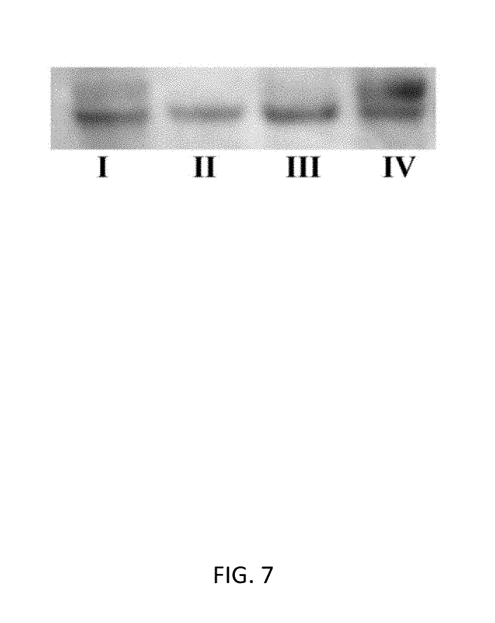

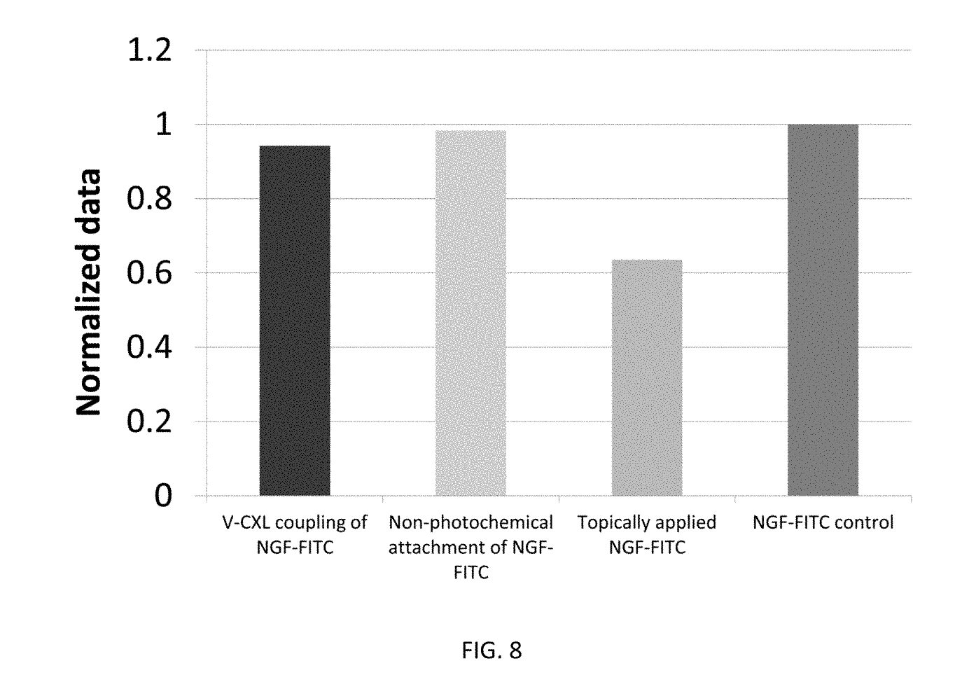

FIG. 7 shows a Western blot detecting applied NGF-FITC within corneal stroma: (I) NGF-FITC control (solution only) (II) topically applied NGF-FITC on corneal stroma, (III) non-photochemical attachment of NGF-FITC to corneal stroma, and (IV) and V-CXL coupling of NGF-FITC to corneal stroma. The presence of the higher molecular weight (MW) band is indicative of binding of the growth factor to the collagen, creating a larger macromolecular complex that is labeled with FITC as a result of the crosslinking. The bar chart shows the normalized band intensity as a function of coupling strategy, showing that V-CXL and non-photochemical crosslinking provides higher NGF surface concentration on corneal stroma than topical delivery alone.

Growth factor release. To understand the ability of growth factors to be liberated from crosslinked collagen, human recombinant EGF (Peprotech) was incorporated within collagen gels during gelation. A neutralized 3% collagen solution with 0.25 mM riboflavin was exposed to blue light (˜458 nm) at 60 mW/cm² for 20 seconds. The formed gel was then cut into cylindrical discs and placed in a PBS solution with or without 0.1% versus 0.2% collagenase at 37° C. on a shaker. The solution was then sampled at intervals starting at 2 hours to 120 hours. An ELISA kit was then used to evaluate the concentration of released EGF in the solution. The results are shown in FIG. 9A. Immersion in PBS alone yields relatively slow release of the growth factor, while exposure to collagenase increases the release in a dose-dependent manner.

Evaluation of V-CXL collagen crosslinking effects. To understand the collateral crosslinking effects of V-CXL, we conducted rheological measurements on collagen gels before and after varying blue light exposure times in the context of constant riboflavin concentration (0.25 mM). Collagen type I was neutralized and mixed with 0.25 mM of riboflavin and exposed to blue light of varying time intervals. Gelation kinetics via V-CXL are shown in FIG. 9B and modulus results summarized in FIG. 9C. The results show a time-dependent substantial increase in modulus of the collagen upon crosslinking by V-CXL compared to collagen solutions alone, which show no change in their mechanical properties. These results imply that the V-CXL coupling process has a stiffening effect on the stromal collagen of the wound bed, as it does with UV-based CXL.

V-CXL crosslinks collagen gels to corneal stroma. A FITC-labelled collagen gels was crosslinked by V-CXL on ex vivo corneal stroma to evaluate its adhesion to corneal collagen. Briefly, FITC-labelled collagen was neutralized and mixed with 0.25 mM riboflavin. Porcine corneas were debrided over an 8 mm circular area and then the FITC-labeled physical collagen gel mixture was applied to the exposed stroma for 10 minutes. The FITC-labeled collagen gels were then exposed to blue light for 20 seconds, quickly forming a crosslinked gel that was adherent to the stroma. The treated corneas were all irrigated aggressively with PBS, and then placed in 4% paraformaldehyde and then sectioned for histological evaluation. The results showed that the V-CXL-crosslinked gel formed a fluorescent membrane on the surface of the stroma (FIG. 9D) while corneas treated with only topical FITC-labeled collagen showed no increased surface fluorescence. These results indicate that the V-CXL reaction facilitates crosslinking between exogenous collagen and endogenous (stromal) collagen. This data serves as further proof of concept that exogenous biomolecules can be bound to the surface of the cornea through protein-protein crosslinks. Gel formation by V-CXL is useful as an alternative method for delivering growth factors to the cornea by encapsulating them within a collagen gel, which is in turn adhered to the wound bed.

FIG. 10A shows live-dead assays showing % living human mesenchymal stem cells (hMSCs) when encapsulated within collagen gels formed by direct exposure to blue light in the presence of different concentrations of riboflavin, showing greater than 90% viability at 72 hours for concentrations of 0.25 mM or less for 20 sec exposure time at 100 mW/cm². FIG. 10 B shows that for the 0.025 mM riboflavin concentration and 20 sec exposure time, greater than 99% of the hMSCs remain viable 1 week after exposure, indicated excellent biocompatibility of the crosslinking regimen in the presence of living cells.

This is consistent with the results of other investigators who have demonstrated the cytocompatibility of blue light-based photochemical crosslinking with riboflavin (Hu et al. (2012) Acta Biomaterialia 8(5):1730-1738). These results bode well for the safety profile of applying these chemistries in situ to wounded and neurotrophic corneas.

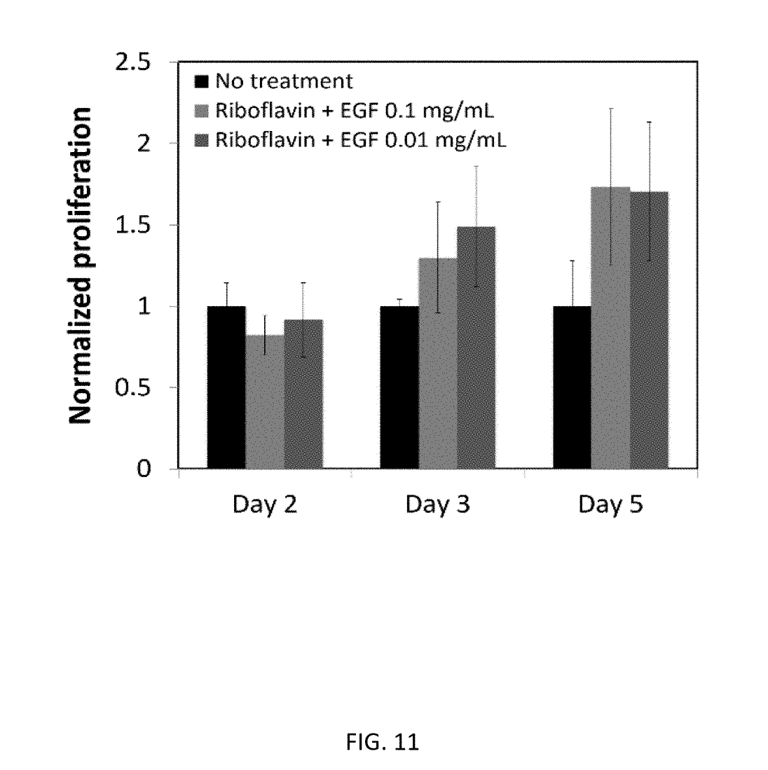

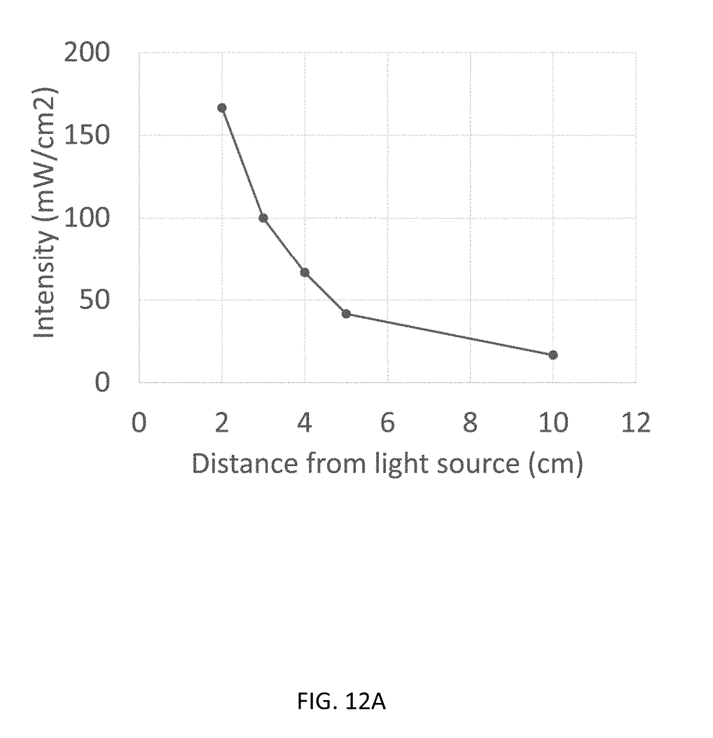

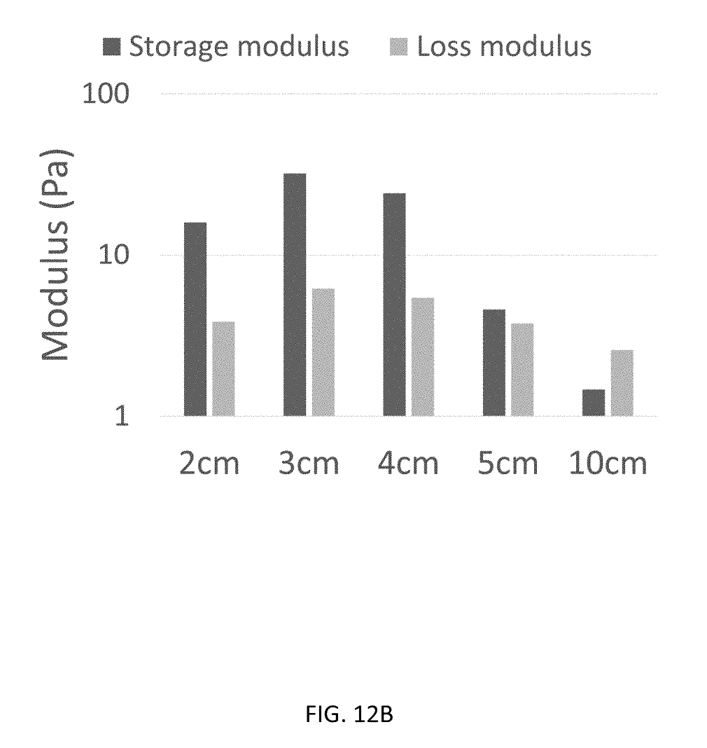

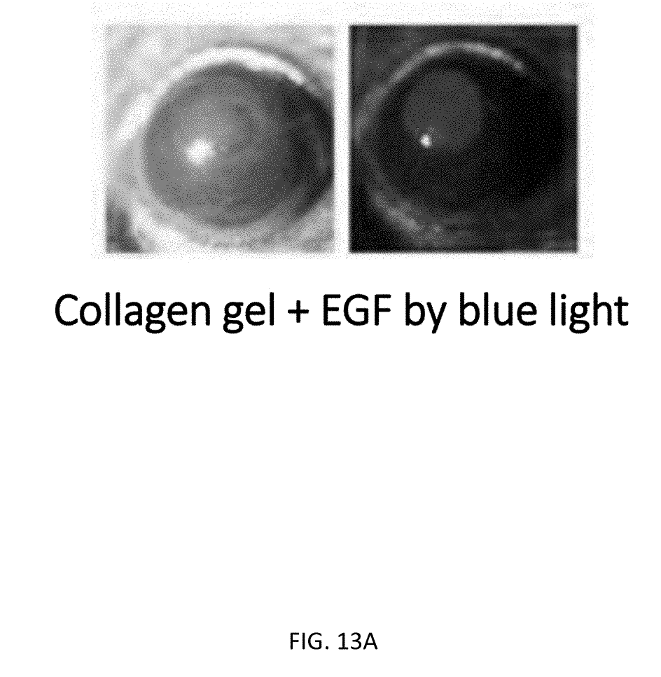

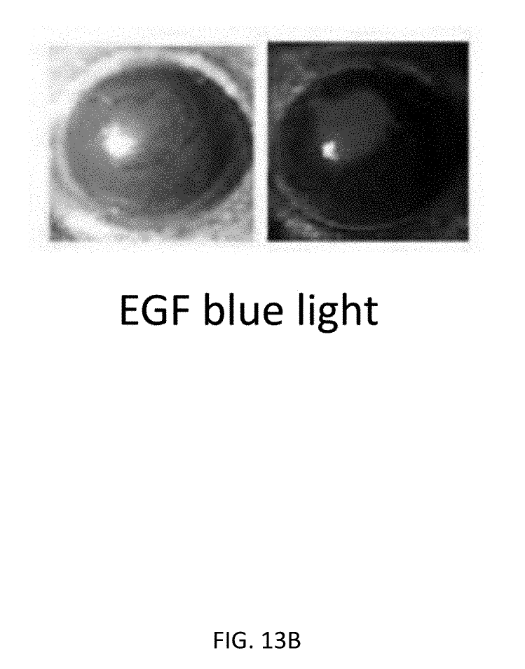

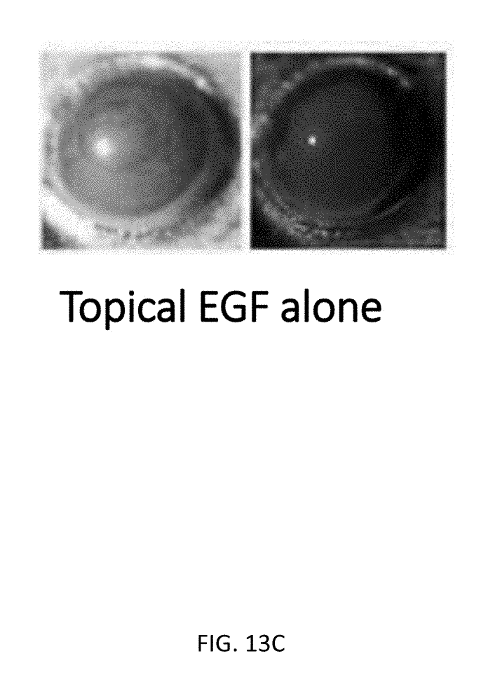

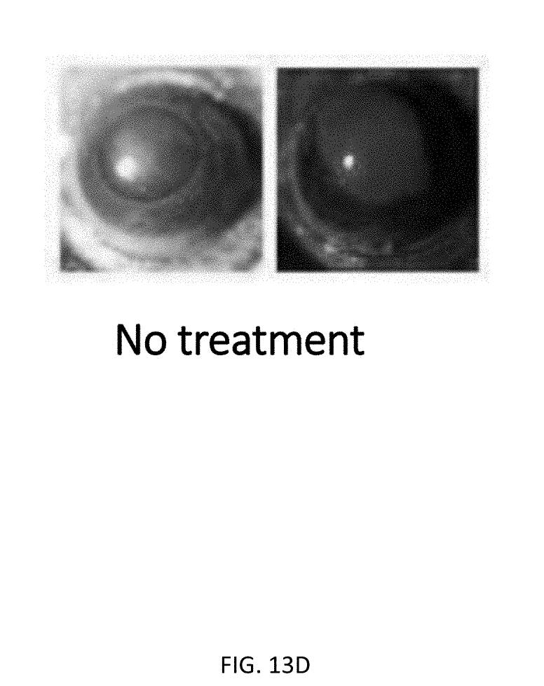

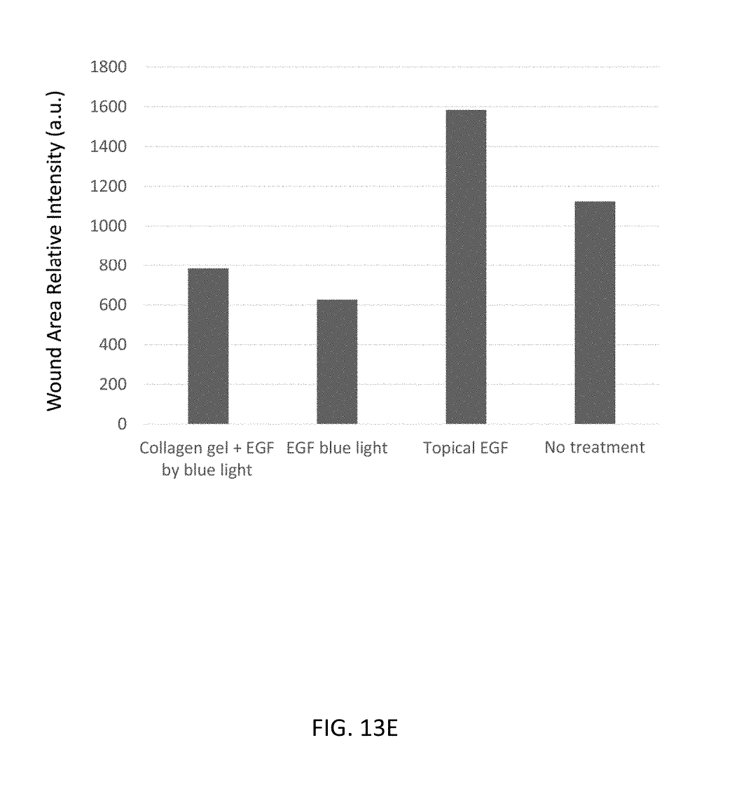

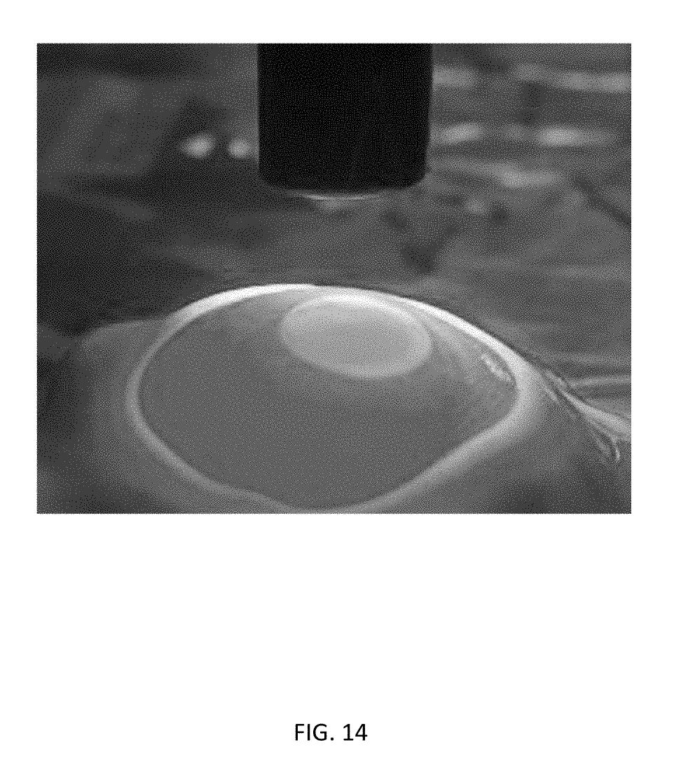

Enhanced cell proliferation on growth factor fortified collagen gels. Late-passage primary rabbit corneal epithelial cells (CECs) of the same cell density were grown on surfaces with EGF bound to collagen using V-CXL as well as standard collagen-coated tissue-culture polystyrene without covalently linked EGF. Greater proliferation of the otherwise senescent CECs was seen over the EGF/collagen gel coatings (both concentrations tested) over 5 days (FIG. 11), indicating that CEC proliferation is enhanced when seeded over collagen with bound growth factors compared to those without bound growth factors. The results indicate that EGF chemically coupled to collagen remains bioactive and stimulates epithelial wound healing.

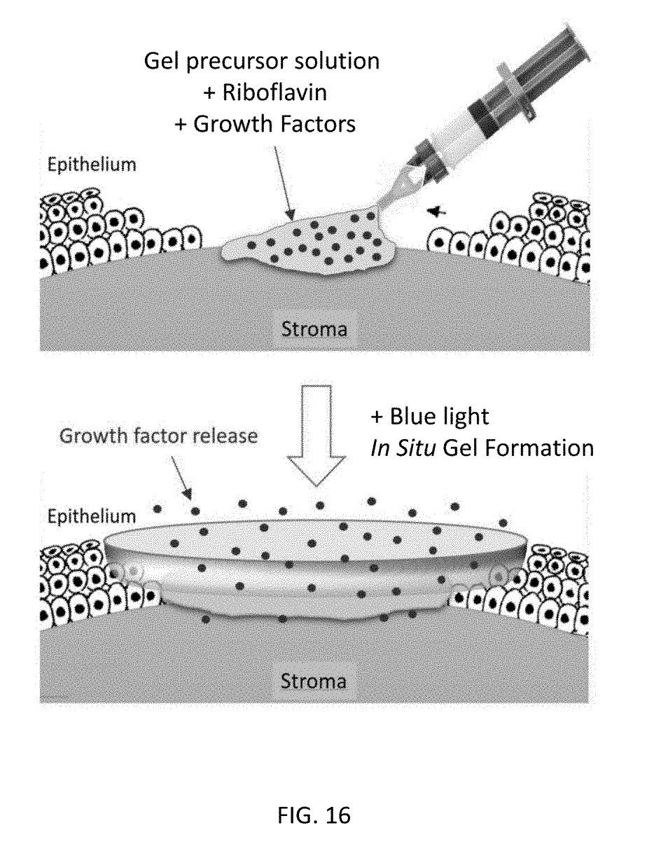

In vivo ocular tolerance and wound healing animal study: In an animal study in rodents, V-CXL was used to bind EGF to wounded corneas. Briefly, a 2 mm diameter debridement was performed, and the wound bed was treated topically with 0.025 mM of riboflavin mixed with 0.01 mg/mL of EGF delivered topically to the wound bed and allowed incubate for 10 minutes followed by 5 seconds of exposure to blue light (˜458 nm) at 60 mW/cm². The eye was then rinsed with BSS. Controls used were topical EGF alone and no treatment. No signs of ocular intolerance such as conjunctival injection, swelling, discharge, or corneal toxicity were observed in the eyes treated with EGF and riboflavin in the V-CXL reaction. Fluorescein staining at 24 hours (FIG. 16) revealed greater reduction in wound area in the EGF-coupled corneas compared with controls (n=2 for all treatments), with complete wound closure on average by 48 hours for all cases. Specifically, ImageJ analysis of the initial results shows that, on average (n=2) at 24 hours, the V-CXL treated eyes had wounds that were approximately 50% of the wound area observed in the topical EGF-treated eyes, and approximately 35% of the wound area of the untreated eyes. These results indicate that V-CXL was well tolerated by the ocular surface, and that coupling of bioactive growth factors to corneal stroma accelerated epithelial wound healing.

### Example 7

### A Wound Treatment System for Riboflavin-Based Corneal Crosslinking with Visible Light Using Growth Factors

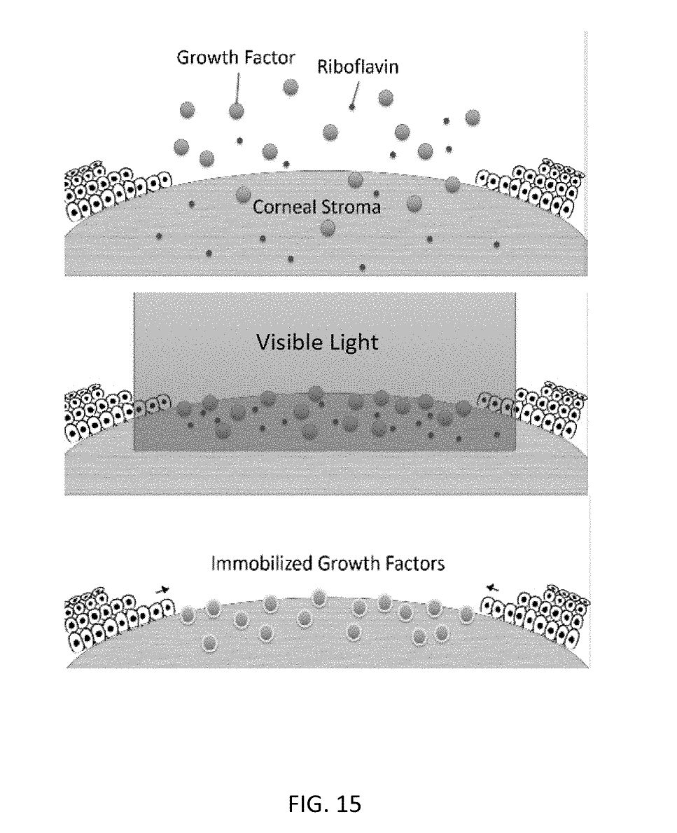

A treatment system for enhancing wound healing composed of (1) an aqueous solution of recombinant growth factor mixed with an FDA-approved photosensitizer (riboflavin), and (2) a blue LED (˜458 nm) light source is shown in FIG. 15. The growth factor/riboflavin solution is applied to a wound bed, followed by exposure to blue light which photoactivates the riboflavin to induce crosslinks between tissue collagen fibrils and adjacent soluble growth factors. The riboflavin solution is then rinsed off, leaving behind growth factor crosslinked within the wound bed extracellular matrix. Blue light is used ubiquitously in ophthalmology to visualize fluorescein staining of corneal wounds (e.g. the “woodslamp” or the blue light used to measure the intraocular pressure through Goldmann applanation tonometry) and also has photo-activating effects on riboflavin to catalyze protein-protein crosslinking (Ibusuki et al. (2007) Tissue Eng. 13(8):1995-2001; Hu et al. (2012) Acta Biomaterialia 8(5):1730-1738).

### Example 8

### A Treatment System for Riboflavin-Based Corneal Crosslinking with Visible Light Using a Growth Factor Eluting Gel

A treatment system for enhancing wound healing composed of (1) an aqueous solution of recombinant growth factor and a gel precursor solution mixed with an FDA-approved photosensitizer (riboflavin), and (2) a blue LED (˜458 nm) light source is shown in FIG. 16. In one example, collagen solution is the gel precursor that is mixed with riboflavin and a growth factor and is exposed to blue light on the surface of a wounded cornea. This leads to gelation of the collagen around the growth factor and adherence of the gel to the corneal stroma. This leads to a sustained release of the growth factors as the applied matrix is broken down and turned over. Other gel precursors such as other proteins such as fibronectin or laminin, or conjugated glycosaminoglycans (e.g. thiolated and methacrylated hyaluronic acid) can be used with any combination of growth factors. This technique can be used in combination with other therapeutic factors such as antibiotic agents, antifibrotic agents, anti-inflammatory agents, chemotherapeutic (anti-oncologic) agents, anti-angiogenic agents, or anti-thrombotic agents, or pro-thrombotic agents.

### Example 9

A Treatment System for Riboflavin-Based Corneal Crosslinking with Visible Light Using Encapsulated Cells

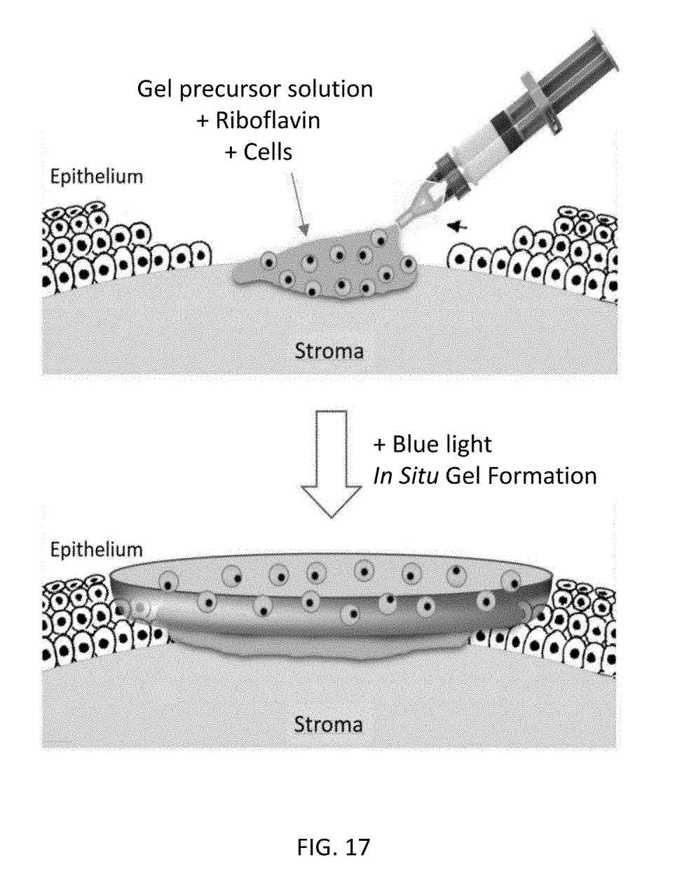

A treatment system for enhancing wound healing composed of (1) an aqueous solution of recombinant growth factor and cells mixed with an FDA-approved photosensitizer (riboflavin), and (2) a blue LED (˜458 nm) light source is shown in FIG. 17. In one example, a collagen solution is the gel pre-cursor that is mixed with riboflavin and hMSCs and is exposed to blue light on the surface of a wounded cornea. This leads to gelation of the collagen around the hMSCs and adherence of the gel to the corneal stroma and the formation of a “living reservoir” of therapeutic factors secreted by the encapsulated hMSCs. Other gel precursors such as other proteins such as fibronectin or laminin, or conjugated glycosaminoglycans (e.g. thiolated and methacrylated hyaluronic acid) can be used with any type of cells, such other forms of stem cells, as well as epithelial, cartilage, bone, liver, cardiac, stromal, endothelial, nerve, corneal, retinal, muscle, and adipose that are appropriate to the damaged tissue being treated. In some cases, this technique can be used as a tissue engineering scaffold for partial or complete regeneration of that tissue. Furthermore, this technique can be used in combination with growth factors or other biomolecules to further stimulate cell growth and/or differentiation.

While the preferred embodiments of the invention have been illustrated and described, it will be appreciated that various changes can be made therein without departing from the spirit and scope of the invention.

What is claimed is:

1. A method of treating damaged tissue in a subject, the method comprising:
a) contacting the damaged tissue with effective amounts of a photosensitizer and one or more therapeutic factors capable of promoting tissue regeneration or repair; and
b) exposing the tissue to light to induce a photocrosslinking reaction, wherein the one or more therapeutic factors are crosslinked directly to the damaged tissue and to one another.

2. The method of claim 1, wherein the one or more therapeutic factors are biomolecules, cells, or a combination thereof.

3. The method of claim 2, wherein the one or more biomolecules are selected from the group consisting of a growth factor, a neurotrophic factor, and an extracellular matrix protein.

4. The method of claim 3, wherein the growth factor is selected from the group consisting of epidermal growth factor and nerve growth factor.

5. The method of claim 2, wherein the extracellular matrix protein is fibronectin, collagen, laminin, fibrin, or derivatives of hyaluronic acid.

6. The method of claim 1, wherein at least one of the one or more therapeutic factors is selected from the group consisting of antibiotic agents, antifibrotic agents, anti-inflammatory agents, chemotherapeutic (anti-oncologic) agents, anti-angiogenic agents, or anti-thrombotic agents, and pro-thrombotic agents.

7. The method of claim 1, wherein said contacting comprises applying the photosensitizer and the one or more therapeutic factors to the damaged tissue at a surface or a sub-surface.

8. The method of claim 1, wherein said contacting comprises applying the photosensitizer and the one or more therapeutic factors to a damaged nerve.

9. The method of claim 1, wherein said contacting comprises applying the photosensitizer and the one or more therapeutic factors simultaneously to the damaged tissue.

10. The method of claim 1, wherein said contacting comprises applying the photosensitizer and the one or more therapeutic factors separately to the damaged tissue.

11. The method of claim 10, wherein the photosensitizer is applied before or after one or more therapeutic factors to the damaged tissue.

12. The method of claim 1, wherein the one or more therapeutic factors are crosslinked simultaneously.

13. The method of claim 1, wherein the tissue is contacted with at least two therapeutic factors separately that are crosslinked sequentially.

14. The method of claim 1, wherein the photosensitizer is riboflavin

15. The method of claim 1, wherein the photosensitizer is coupled to at least one biomolecule and is capable of forming a covalent linkage with tissue upon exposure to light.

16. The method of claim 1, wherein said contacting comprises applying the one or more therapeutic factors to the damaged tissue in a pattern, tracks, or gradient.

17. The method of claim 16, wherein the gradient is a gradient of growth factors or axon guidance factors.

18. The method of claim 16, wherein the gradient is produced by varying light intensity, length of light exposure, or concentration of therapeutic factors along the damaged tissue.

19. The method of claim 1, wherein said contacting comprises applying a composition comprising the one or more therapeutic factors to the damaged tissue.

20. The method of claim 19, wherein the composition further comprises the photosensitizer.

21. The method of claim 20, wherein the composition further comprises a pharmaceutically acceptable excipient.

22. The method of claim 21, wherein the pharmaceutically acceptable excipient is a thickening agent.

23. The method of claim 21, wherein the composition is a solution or gel.

24. The method of claim 21, wherein the photocrosslinking reaction changes the viscosity of the composition.

25. The method of claim 1, wherein said contacting comprises applying a wound dressing comprising the one or more therapeutic factors to the damaged tissue.

26. The method of claim 25, wherein the wound dressing comprises a gel, a viscoelastic solution, putty, a physical matrix or a membrane.

27. The method of claim 1, wherein the photosensitizer further comprises a non-light-activatable crosslinking moiety.

28. The method of claim 27, further comprising crosslinking one or more therapeutic factors with the photosensitizer using the non-light-activatable crosslinking moiety to produce light-activatable bioconjugates of the therapeutic factors.

29. The method of claim 28 wherein the light-activatable bioconjugates of the therapeutic factors comprise thiolated hyaluronic acid and methacrylated hyaluronic acid, wherein the thiolated hyaluronic acid and the methacrylated hyaluronic react to form a gel upon exposure to blue light in the presence of riboflavin.

30. The method of claim 1, wherein said treating accelerates healing of the damaged tissue.

31. The method of claim 1, wherein said treating increases thickness of an epithelial layer of the damaged tissue, increases rate of epithelialization of the damaged tissue, promotes nerve regeneration in the damaged tissue, or shortens time required for wound closure in the damaged tissue.

32. The method of claim 1, wherein the tissue damage comprises a diabetic ulcer, a neurotrophic ulcer, a burn, a chemical injury, or a nerve injury

33. The method of claim 1, wherein the subject is human.

34. The method of claim 1, further comprising preparing the damaged tissue prior to treating the subject by exfoliation or debridement of fibrotic or necrotic tissue.

35. The method of claim 1, further comprising administering one or more therapeutic agents to the subject.

36. The method of claim 35, wherein the one or more therapeutic agents are selected from the group consisting of an antiseptic agent, an analgesic agent, an anti-inflammatory agent, and an anesthetic.

37. The method of claim 1, further comprising administering a cellular therapy to the damaged tissue.

38. The method of claim 37, wherein the cellular therapy is allogeneic cell therapy, autologous cell therapy, or stem cell therapy.

39. The method of claim 1, wherein multiple cycles of treatment are administered to the subject for a time period sufficient to effect at least a partial healing of the damaged tissue.

40. The method of claim 39, wherein multiple cycles of treatment are administered to the subject for a time period sufficient to effect a complete healing of the damaged tissue.

41. The method of claim 1, wherein the light is visible light or ultraviolet light.

42. The method of claim 41, wherein the visible light is blue light.

43. The method of claim 1, wherein the damaged tissue is corneal tissue.

44. The method of claim 43, wherein the one or more therapeutic factors are selected from the group consisting of epidermal growth factor (EGF) and nerve growth factor.

45. The method of claim 43, further comprising applying a bandage contact lens to the cornea after the photocrosslinking reaction.

46. The method of claim 43, wherein the subject has corneal tissue damage caused by neurotrophic keratopathy, recurrent corneal erosion, a corneal ulcer, exposure keratopathy, or physical trauma.

47. A tissue repair system comprising:
a) an ultraviolet or a visible light source;
b) one or more biomolecules selected from the group consisting of epidermal growth factor (EGF) and nerve growth factor (NGF), and
c) a photosensitizer.

48. The tissue repair system of claim 47, further comprising a light filter.

49. The tissue repair system of claim 47, further comprising a topical applicator or dispenser.

50. The tissue repair system of claim 47, wherein the photosensitizer is covalently coupled to a biomolecule.

51. The tissue repair system of claim 47, wherein the visible light source provides blue light.

52. The tissue repair system of claim 47, wherein the photosensitizer is riboflavin,

53. A kit comprising the tissue repair system of claim 47, further comprising instructions for treating damaged tissue.

54. A photosensitizer and at least one therapeutic factor for use in treating damaged tissue in a subject by photocrosslinking the at least one therapeutic factor directly onto the damaged tissue.
